(19) United States
(12) Patent Application Publication
(10) Pub. No.: US 2025/0255851 A1
(43) Pub. Date: Aug. 14, 2025

(54) SOLID FORMS OF AN ALDOSTERONE SYNTHASE INHIBITOR

(71) Applicant: Boehringer Ingelheim International GmbH, Ingelheim am Rhein (DE)

(72) Inventors: Brian J. LINEHAN, Cheshire, CT (US); John Andrew SMOLIGA, Brookfield, CT (US); Zheng Jane LI, Kendall Park, NJ (US)

(21) Appl. No.: 19/051,292

(22) Filed: Feb. 12, 2025

Related U.S. Application Data
(60) Provisional application No. 63/553,234, filed on Feb. 14, 2024.

(51) Int. Cl. A61K 31/4188; A61K 31/7048; C07D 491/052

(52) U.S. Cl. CPC A61K 31/4188; A61K 31/7048; C07D 491/052

(57) ABSTRACT

Disclosed are solid forms of an inhibitor of aldosterone synthase (ASi) having the formula (1)

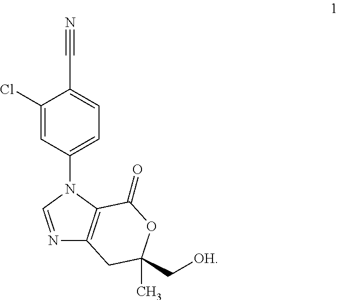 [SMILES: C[C@]1(CO)Cc2ncn(-c3ccc(C#N)c(Cl)c3)c2C(=O)O1]

The invention also relates to methods of making these solid forms, pharmaceutical compositions comprising these solid forms, and their use for medical conditions responsive to treatment with an inhibitor of aldosterone synthase.

### FIELD OF THE INVENTION

[symbol] inventions relate to solid forms of an inhibitor of aldosterone synthase (AS). The invention also relates to methods of making these solid forms, pharmaceutical compositions comprising these solid forms, and their use for medical conditions responsive to treatment with an inhibitor of AS.

### BACKGROUND OF THE INVENTION

Compound 1 is an aldosterone synthase inhibitor (ASi) and has the structure shown below:

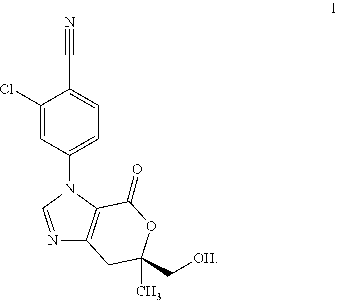 [SMILES: C[C@]1(CO)Cc2ncn(-c3ccc(C#N)c(Cl)c3)c2C(=O)O1]

The name of Compound 1 is 2-chloro-4-[(6R)-6-(hydroxymethyl)-6-methyl-4-oxo-3H,4H,6H,7H-pyrano[3,4-d]imidazol-3-yl]benzonitrile. Compound 1 is also known as vicadrostat.

Compound 1 is useful for treating kidney-related disorders such as diabetic nephropathy; non-diabetic kidney disease including glomerulosclerosis, glomerulonephritis, IGA nephropathy, nephritic syndrome and focal segmental glomerulosclerosis (FSGS); cardiovascular diseases including hypertension, pulmonary arterial hypertension, Conn's syndrome, systolic heart failure, diastolic heart failure, left ventricular dysfunction, left ventricular stiffness and fibrosis, left ventricular filing abnormalities, arterial stiffness, atherosclerosis and cardiovascular morbidity associated with primary or secondary hyperaldosteronism; adrenal hyperplasia and primary and secondary hyperaldosteronism. (See, e.g., WO 2016/014736.) Compound 1 is currently under development for the treatment of chronic kidney disease (CKD) in patients with and without diabetes. (See ClinicalTrials.gov Identifier: NCT05182840.)

The preparation of Compound 1 is described in WO 2016/014736 (see compound 29A). According to WO 2016/014736, a crystalline form of Compound 1 was obtained, but the reference does not describe any method of preparing the solid form or any of its characteristics.

Because of its beneficial properties as a therapeutic, Applicant carried out further studies directed to crystalline forms of Compound 1 having advantageous pharmaceutical properties such as, for example, processability, stability, and solubility.

### BRIEF SUMMARY OF THE INVENTION

The invention relates to novel solid forms of the Compound 1 (herein, collectively “the compounds of the invention”).

The invention also relates to methods of making the compounds of the invention and their use as inhibitors of AS.

In a further aspect, the present invention relates to pharmaceutical compositions, comprising a compound of the invention, optionally together with one or more inert carriers and/or diluents.

A further aspect of the present invention relates to compounds of the invention or pharmaceutical compositions comprising the compounds of the invention for the use in the prevention and/or treatment of metabolic and cardiovascular disorders.

In one embodiment, the present invention relates to methods for preventing, slowing the progression of, delaying or treating diseases or conditions which can be influenced by inhibition of AS, such as chronic kidney disease, diabetic kidney disease, heart failure, heart failure with reduced ejection fraction (HFrEF), heart failure with preserved ejection fraction (HFpEF), heart failure with left ventricular ejection fraction≥40% (LVEF≥40%), heart failure with LVEF<40%, and resistant hypertension (rHPT), comprising administering a pharmaceutically effective amount of a compound of the invention to a patient in need thereof. The methods of the invention also encompass pharmaceutical composition comprising the compounds of the invention for the treatment of the corresponding diseases or disorders described herein.

In another embodiment, the present invention relates to compounds of the invention or pharmaceutical compositions comprising said compounds for use in preventing, slowing the progression of, delaying or treating diseases or conditions which can be influenced by inhibition of AS, such as chronic kidney disease, diabetic kidney disease, heart failure, heart failure with reduced ejection fraction (HFrEF), heart failure with preserved ejection fraction (HFpEF), heart failure with left ventricular ejection fraction≥40% (LVEF≥40%), heart failure with LVEF<40%, and resistant hypertension (rHPT). The use comprises the manufacture of medicaments for the treatment of the corresponding diseases or disorders described herein.

In one embodiment, the invention relates to crystalline forms of Compound 1 as described in Table 1 (“the compounds of the invention”) or mixtures thereof.

TABLE 1
Crystalline forms of the compounds of the invention.

| Solid Form | Molecular Formula |
|---|---|
| Form I | C₁₅H₁₂ClN₃O₃ |
| Form II | C₁₅H₁₂ClN₃O₃ |
| Form III | C₁₅H₁₂ClN₃O₃ |
| Form IV | C₁₅H₁₂ClN₃O₃•CH₃CN |
| | Acetonitrile solvate |
| Form VI | C₁₅H₁₂ClN₃O₃•ACE |
| | Acetone solvate |
| Form VII | C₁₅H₁₂ClN₃O₃•THF |
| | Tetrahydrofuan solvate |
| Form IX | C₁₅H₁₂ClN₃O₃•0.5TOL |
| | Hemi-toluene solvate |
| Form X | C₁₅H₁₂ClN₃O₃ |

One embodiment of the invention relates to crystalline Form I of Compound 1.

In another embodiment, the invention relates to crystalline Form II of Compound 1.

In another embodiment, the invention relates to crystalline Form III of Compound 1.

In another embodiment, the invention relates to crystalline Form IV of Compound 1.

In another embodiment, the invention relates to crystalline Form VI of Compound 1.

In another embodiment, the invention relates to crystalline Form VII of Compound 1.

In another embodiment, the invention relates to crystalline Form IX of Compound 1.

In another embodiment, the invention relates to crystalline Form X of Compound 1.

In yet another embodiment, the invention relates to a mixture of crystalline Form I and crystalline Form X of Compound 1.

In yet another embodiment, the invention relates to methods of making the crystalline forms of the compounds of the invention.

### BRIEF DESCRIPTION OF THE FIGURES

FIG. 1A is an X-ray powder diffraction (XRPD) pattern of Form I of Compound 1.

FIG. 1B is a ¹³C ssNMR spectrum of Form I of Compound 1.

FIG. 1C is a thermal analysis profile of Form I of Compound 1 determined by DSC measurement.

FIG. 1D is a thermal analysis of Form I of Compound 1 determined by TGA.

FIGS. 1E-1 shows a Dynamic Vapor Sorption (DVS) isotherm plot for cycle 1 of Form I of Compound 1.

FIG. 1E-2 shows a DVS isotherm plot for cycle 2 of Form I of Compound 1.

FIG. 2A is an X-ray powder diffraction (XRPD) pattern of Form X of Compound 1.

FIG. 2B is a ¹³C ssNMR spectrum of Form X of Compound 1.

FIG. 2C is a thermal analysis profile of Form X of Compound 1 determined by DSC measurement.

FIG. 2D is a thermal analysis of Form X of Compound 1 determined by TGA.

FIG. 2E shows a 2-cycle DVS isotherm plot for Form X of Compound 1.

FIG. 3A is an X-ray powder diffraction (XRPD) pattern of Form II of Compound 1.

FIG. 3B is a thermal analysis profile of Form II of Compound 1 determined by DSC measurement.

FIG. 3C is a thermal analysis of Form II of Compound 1 determined by TGA.

FIG. 4A is an X-ray powder diffraction (XRPD) pattern of Form III of Compound 1.

FIG. 4B is a thermal analysis profile of Form III of Compound 1 determined by DSC measurement.

FIG. 4C is a thermal analysis of Form III of Compound 1 determined by TGA.

FIG. 5A is an X-ray powder diffraction (XRPD) of Form IV Compound 1.

FIG. 5B is a thermal analysis profile of Form IV of Compound 1 determined by DSC measurement.

FIG. 5C is a thermal analysis of Form IV of Compound 1 determined by TGA.

FIG. 6A is an X-ray powder diffraction (XRPD) of Form VI Compound 1.

FIG. 6B is a thermal analysis profile of Form VI of Compound 1 determined by DSC measurement.

FIG. 6C is a thermal analysis of Form VI of Compound 1 determined by TGA.

FIG. 6D shows a DVS isotherm plot for Form VI of Compound 1

FIG. 7A is an X-ray powder diffraction (XRPD) of Form VII Compound 1.

FIG. 7B is a thermal analysis profile of Form VII of Compound 1 determined by DSC measurement.

FIG. 7C is a thermal analysis of Form VII of Compound 1 determined by TGA.

FIG. 7D shows a DVS isotherm plot for Form VII of Compound 1

FIG. 8A is an X-ray powder diffraction (XRPD) of Form IX Compound 1.

FIG. 8B is a thermal analysis profile of Form IX of Compound 1 determined by DSC measurement.

FIG. 8C is a thermal analysis of Form IX of Compound 1 determined by TGA

FIG. 9 is an X-ray powder diffraction (XRPD) of an amorphous form of Compound 1.

### DETAILED DESCRIPTION OF THE INVENTION

### Abbreviations

ACN Acetonitrile
ACE Acetone
AS Aldosterone synthase
DSC Differential scanning calorimetry
DVS Dynamic Vapor Sorption
HFpEF Heart failure with preserved ejection fraction
HFrEF Heart failure with reduced ejection fraction
LVEF Left ventricular ejection fraction
MEK Methylethyl ketone or 2-Butanone
MIBK Methylisobutyl ketone
O.D. Outside diameter
RH Relative humidity
rHPT Resistant hypertension
SGLT2 Sodium-glucose cotransporter-2
ssNMR Solid-state nuclear magnetic resonance
TGA Thermogravimetric Analysis
THF Tetrahydrofuran
TOL Toluene
XRPD X-ray powder diffraction

As discussed above, the invention relates to crystalline forms of Compound 1 as described in Table 1 including mixtures of these crystalline forms. The invention also relates to compositions comprising the crystalline compounds of the invention and the use of such compounds and compositions for treating diseases or disorders that are responsive to treatment with an inhibitor of AS.

In one embodiment, the invention relates to mixtures comprising Form I and any other form of Compound 1 including, but not limited to, the other solid forms described in Table 1. In other embodiments, the invention relates to mixtures comprising Form X and any other form of Compound 1 including, but not limited to, the other solid forms described in Table 1.

In another, the invention relates to mixtures comprising Form II of Compound 1 and any other form of Compound 1 including, but not limited to, the other solid forms described in Table 1.

In another, the invention relates to mixtures comprising Form III of Compound 1 and any other form of Compound 1 including, but not limited to, the other solid forms described in Table 1.

In another, the invention relates to mixtures comprising Form IV of Compound 1 and any other form of Compound 1 including, but not limited to, the other solid forms described in Table 1.

In another, the invention relates to mixtures comprising Form VI of Compound VI and any other form of Compound 1 including, but not limited to, the other solid forms described in Table 1.

In another, the invention relates to mixtures comprising Form VII of Compound 1 and any other form of Compound 1 including, but not limited to, the other solid forms described in Table 1.

In another, the invention relates to mixtures comprising Form IX of Compound 1 and any other form of Compound 1 including, but not limited to, the other solid forms described in Table 1.

WO 2016/014736 describes the preparation of a racemic mixture comprising Compound 1 (referred to as 29A) and its enantiomer (referred to as 29B). The WO 2016/014736 publication also describes the separation of the two enantiomeric forms using chiral chromatography. According to the WO 2016/014736 publication, the absolute stereochemistries for the two enantiomers were determined using high resolution single crystal X-ray crystallography. However, the WO 2016/014736 publication makes no mention how the crystalline form 29A was prepared; the elemental analysis of the crystalline form 29A; or any characteristics of the crystalline form 29A such as XRPD, ssNMR, thermal analysis, or stability (for example, resistance to change in crystalline form). Likewise, the WO 2016/014736 publication makes no mention of any bulk characteristics of the crystalline form 29A such as, for example, flowability, friability, or bulk density.

Applicant found the crystalline forms of the compounds of the invention are useful for pharmaceutical preparations or formulations containing solid forms of Compound 1. Form I and Form X have particularly advantageous properties for such pharmaceutical preparations or formulations. These desirable properties of Form I and Form X include, for example, low hygroscopicity and thermal stability; and desirable bulk solid properties such as flow rate, bulk density, and resistance to mechanical attrition. Both Form I and Form X meet the requirements of class 1 compounds according to the Biopharmaceutics Classification System (BCS).

Applicant found that the solvates of Compound 1 (Form IV, Form VI, Form VII and Form IX) may also be used for formulations containing solid forms of Compound 1. However, these solvate of Compound 1 are generally not as stable (for example, resistant to change in crystalline form) as Form I and Form X. This lack of stability generally requires special processing and storage conditions for the solvates, thus making them less desirable as a component of a solid form of a pharmaceutical preparation. However, each of Form IV, Form VI, Form VII and Form IX can be used as a starting material for preparing Form I or Form X. For example, Form IV, Form VI, Form VII and Form IX convert to Form I and/or Form X at elevated temperature and/or under high humidity. Alternatively, processes for preparing Compound 1 may involve formation of a crystalline solvate form of Compound 1 as an intermediate step prior to final preparation of Form I or Form X. For example, the purification and/or isolation of the crystalline solvates of Compound 1 may be used to remove impurities prior to conversion of the crystalline solvates to Form I and/or Form X using methods described herein.

In one embodiment, the invention relates to Form I that is substantially free of any other form of Compound 1. As used herein, “substantially free” means that the solid compound contains at least about 75% of Form I of Compound 1 based on total molar amounts of any other form of Compound 1. The amount of any form of the Compound 1 that may be present in Form I can be determined, for example, using the methods described herein.

In another embodiment, the invention relates to Form X that is substantially free of any other form of Compound 1 including the amorphous form and other crystalline forms including Form I. As used herein, “substantially free” means that the solid compound contains at least about 75% of Form X of Compound 1 based on total molar amounts of any other form of Compound 1. The amount of any form of the Compound 1 that may be present in Form X can be determined, for example, using the methods described herein.

In another embodiment, the invention relates to a mixture of Form I and Form X that is substantially free of any other form of Compound 1. As used herein, “substantially free” means that the solid compound contains at least about 75% of Form I and Form X of Compound 1 based on total molar amounts of any other form of Compound 1. The amount of any form of the Compound 1 that may be present in Form I can be determined, for example, using the methods described herein.

### Characterization

The compounds of the invention can be characterized by the methods described below. Methods of preparing the compounds of the invention are described in the Experimental section.

### X-Ray Powder Diffraction (XRPD)

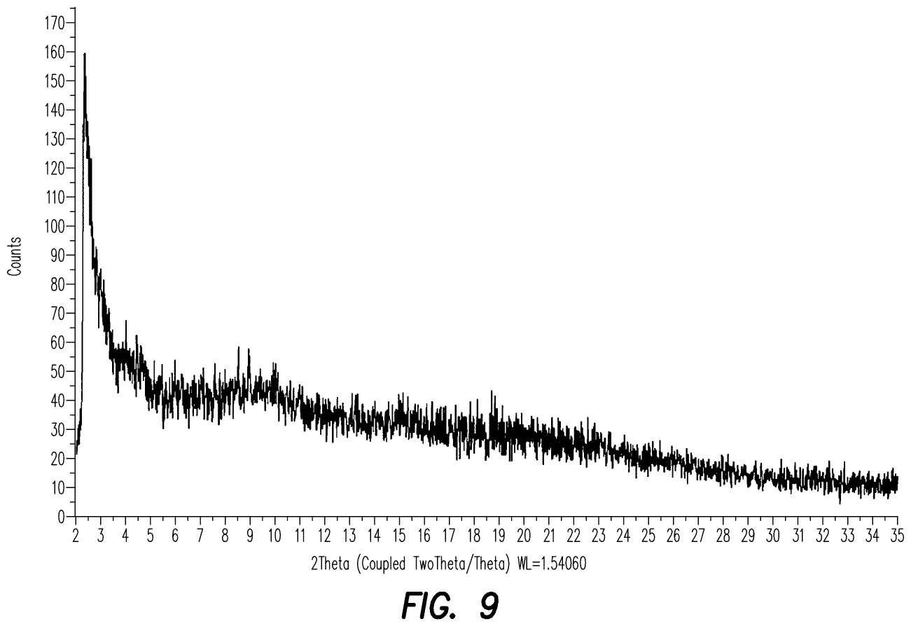

XRPD is performed with a Bruker AXS X-Ray Powder Diffractometer Model D8 Advance, using CuKa radiation (1.54A) in parafocusing mode with a graphite monochromator and a scintillation detector. Each pattern is obtained by scanning over a range of 2 degrees-35 degrees 2T, step size of 0.05 degrees 2T, step time of 1 sec per step. Exemplary XRPD spectra of the compounds of the invention are found in the Figures. An exemplary XRPD spectrum of an amorphous form of Compound 1 is shown in FIG. 9. The X-ray powder diffraction (XRPD) characteristics for the compounds of the invention reported herein have a standard deviation of ±0.2 2θ.

¹³C Solid-State NMR (ssNMR)

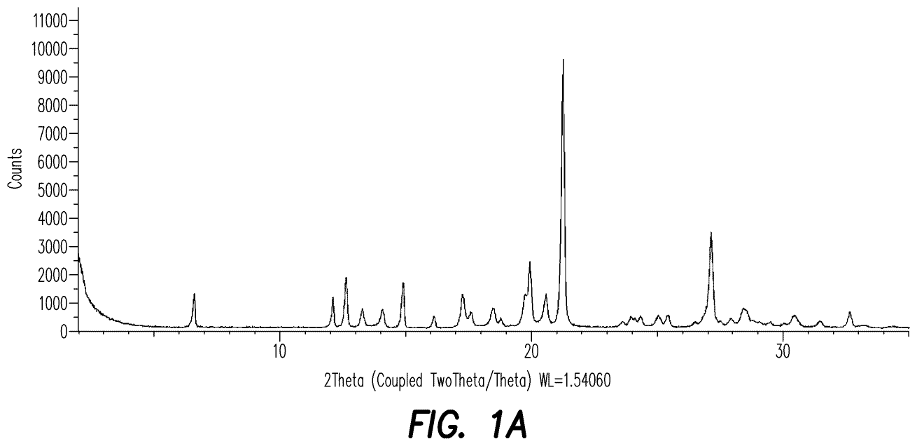

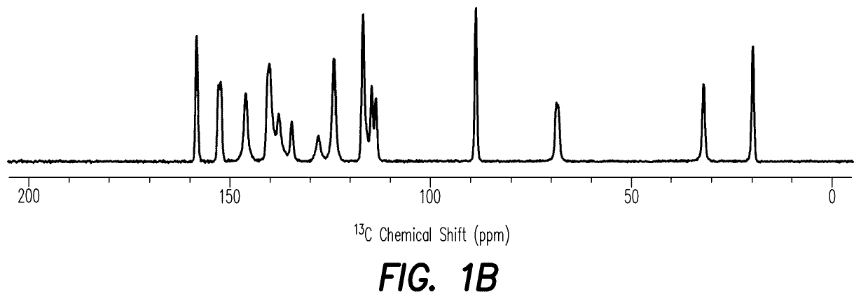

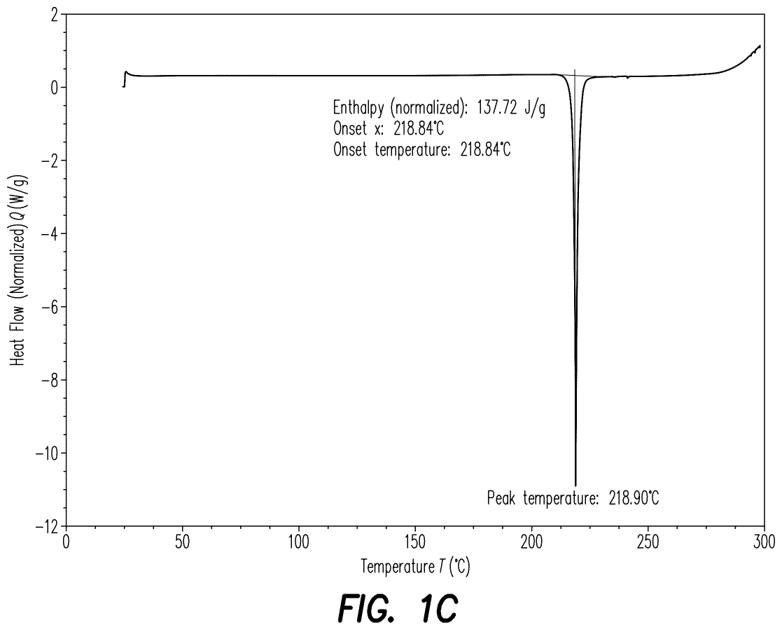

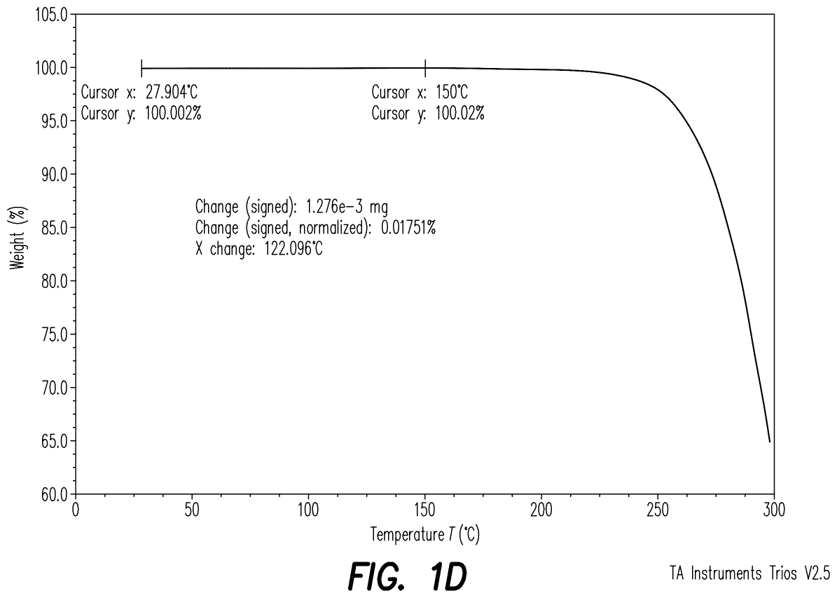

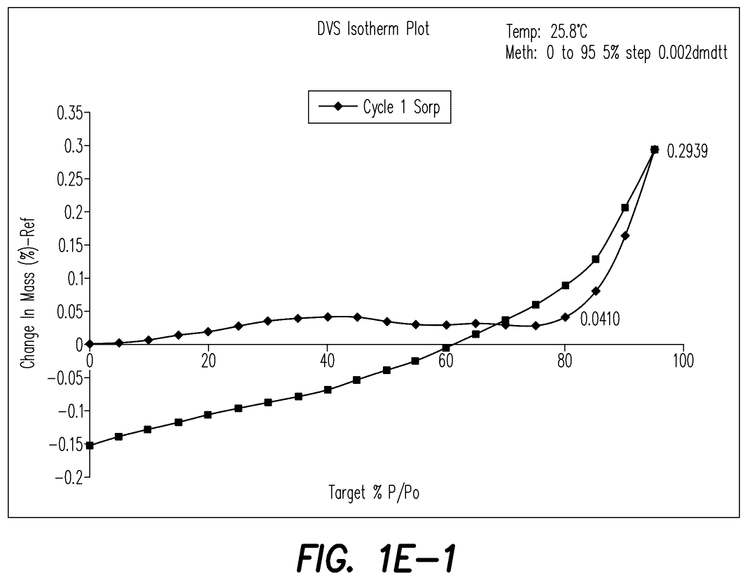

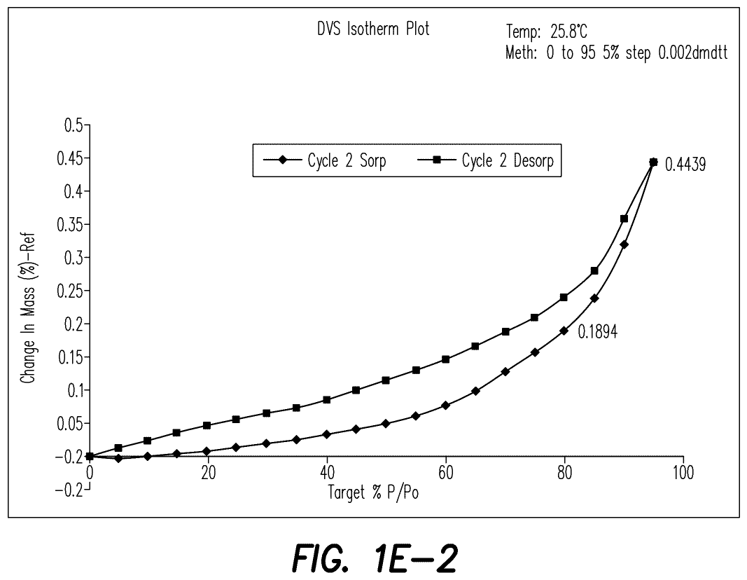

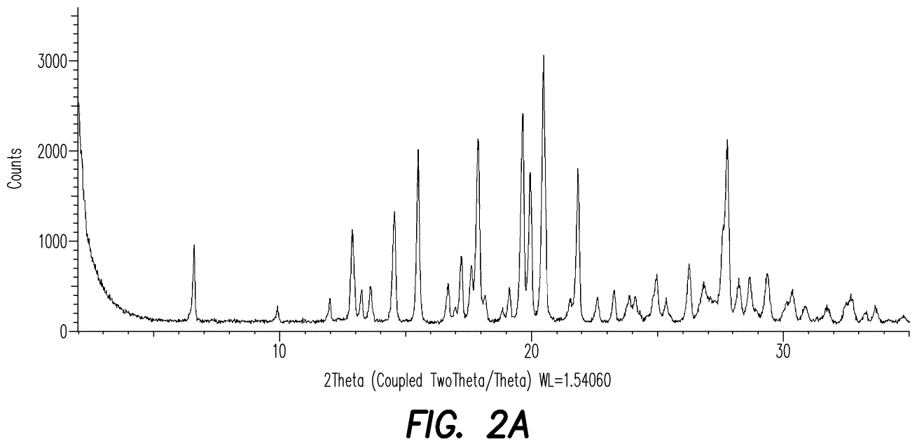

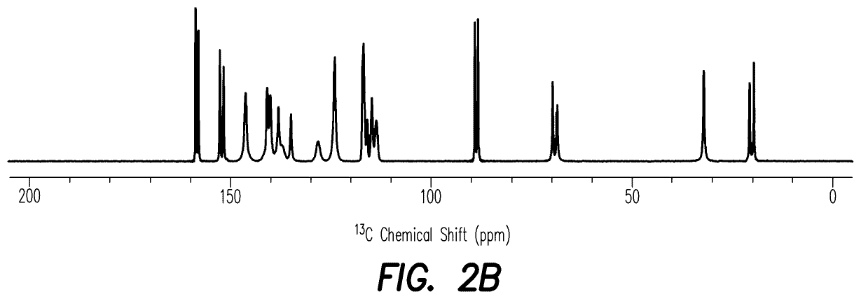

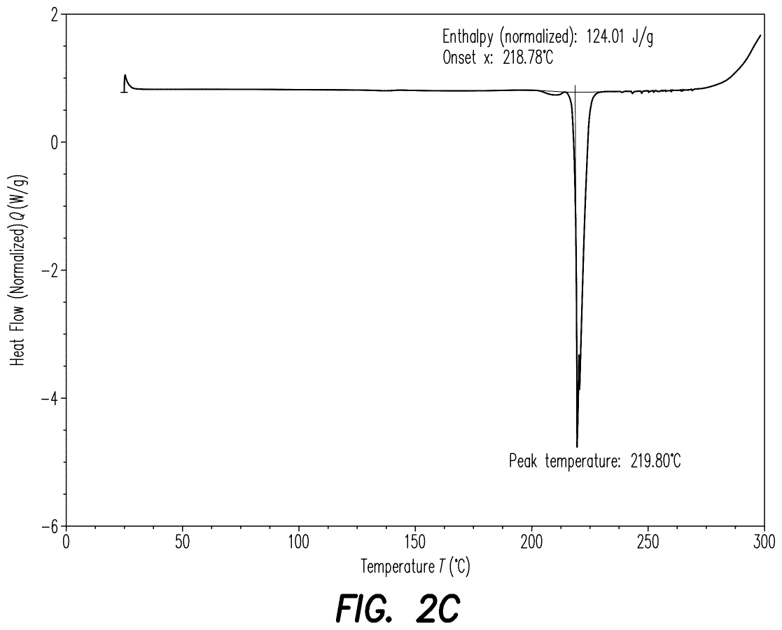

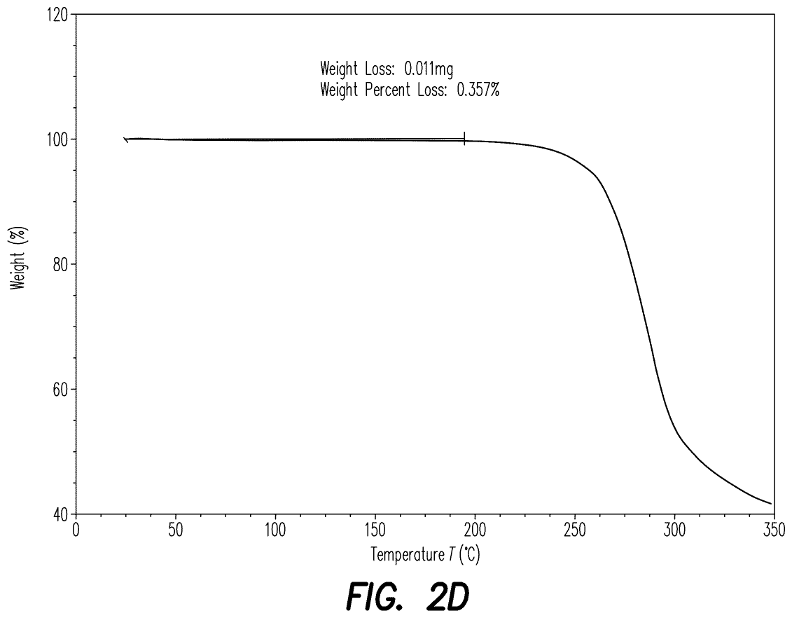

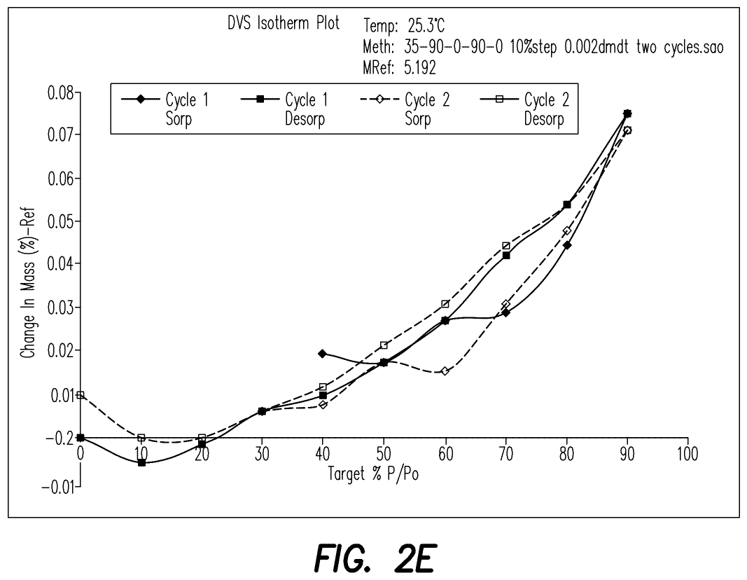

¹³C Solid-state NMR (ssNMR) data are acquired on a 500 MHz Bruker Avance III HD NMR spectrometer (Bruker Biospin, Inc., Billerica, MA) at 11.7 T (¹H=500.28 MHz, ¹³C=125.81 MHz). Samples are packed in 4 mm O.D. zirconia rotors with Kel-F(R) drive tips. A Bruker model BL4 VTN probe is used for data acquisition and sample spinning about the magic-angle (54.74 degrees). Sample spectrum acquisition uses a spinning rate of 12 kHz. A standard cross-polarization pulse sequence is used with a ramped Hartman-Hahn match pulse on the proton channel at ambient temperature and pressure. The pulse sequence uses a 4 millisecond contact pulse and a 20, 3, 10, 3.64 second recycle delay for Form I, Form III (hydrate), Form IV (ACN/H₂O solvate), and Form V (hydrate), respectively. SPINAL64 decoupling and TOSS sideband suppression are also employed in the pulse sequence. No exponential line broadening is used prior to Fourier transformation of the free induction decay. Chemical shifts are referenced using the secondary standard of adamantane, with the high frequency resonance being set to 38.48 ppm. The magic-angle is set using the ⁷⁹Br signal from KBr powder at a spinning rate of 5 kHz. Exemplary ¹³C ssNMR spectra of Form I and Form X are found in FIGS. 1B and 2B, respectively, and in the Tables below. The reported chemical shifts have an uncertainty of ±0.3 ppm.

### Differential Scanning Calorimetry (DSC)

DSC analysis is performed with a differential scanning calorimeter (Q2500, TA instruments, New Castle, DE), using the following general procedure. About 5 mg of powder is weighed into a crimped aluminum pan with pin hole. The sample is heated at 10° C./min from room temperature to 300° C. using the Q2500 DSC. Exemplary DSC traces of the compounds of the invention are found in in the Figures. Results are reported below.

### Thermal Gravimetric Analysis (TGA)

TGA analysis is performed with a Thermogravimetric Analyzer (TGA5500, TA instruments, New Castle, DE), using the following general procedure. About 5-10 mg of powder is weighed into an open aluminum pan. The sample is heated at 10° C./min from room temperature to 350° C. using the TGA5500 TGA. Exemplary TGA traces of the compounds of the invention are found in the FIGS. 1D, 2D, 3D and 4D. Results are reported below.

### Dynamic Vapor Sorption (DVS)

Water sorption isotherms are determined using a dynamic vapor sorption system (Advantage 1, DVS, London, UKDVS Intrinsic Plus, Surface Measurement Systems, Allentown, PA). Approximately 5-10 mg of solid is weighed into a tared aluminum pan. The samples are subjected to 0 to 90% RH stepwise with a step size of 510% at 25° C. Equilibration criteria are dm/dt of 0.002% over 5 minutes or 360 minutes at a specified % RH. Each sample is equilibrated at each RH step for at least 60 min, and equilibrium is assumed if weight increase is less than 0.1% within one minute, and the maximum duration on each RH is 6 hours. Therefore, each sample is held at a given RH for 1 to 6 hours depending on how fast the equilibrium is reached. Exemplary DVS of the compounds of the invention are found in the Figures.

### Characteristics of Form I

The X-ray powder diffraction (XRPD) pattern of Form I of Compound 1 is shown in FIG. 1A; the ¹³C solid state NMR spectrum of Form I of Compound 1 is shown in FIG. 1B; the thermal analysis profiles of Form I of Compound 1 determined by DSC and TGA measurement are shown in FIGS. 1C and 1D; respectively, and the DVS profile is shown in FIG. 1E.

Characteristic XRPD peaks and ¹³C solid-state nuclear magnetic resonance peaks are provided in Table 2 and Table 3, respectively.

TABLE 2
X-ray powder diffraction (XRPD) characteristics
from FIG. 1A for Form I.

| 2Θ, | Intensity I/I, |
|---|---|
| [°] | [%] |
| 4.4 | 2.1 |
| 6.6 | 10.3 |
| 12.1 | 9.5 |
| 12.6 | 16.8 |
| 13.3 | 6.4 |
| 14.1 | 6.5 |
| 14.9 | 14.9 |
| 16.1 | 3.9 |
| 17.3 | 12.3 |
| 17.6 | 5.2 |
| 18.5 | 7.0 |
| 18.8 | 2.6 |
| 19.9 | 23.0 |
| 20.6 | 11.9 |
| 21.3 | 100.0 |
| 23.6 | 1.8 |
| 24.0 | 3.6 |
| 24.3 | 3.7 |
| 25.0 | 4.1 |
| 25.4 | 4.1 |
| 26.5 | 1.7 |
| 27.1 | 34.7 |
| 27.9 | 2.9 |
| 28.5 | 6.8 |
| 29.1 | 1.4 |
| 29.5 | 1.4 |
| 30.5 | 4.2 |
| 31.5 | 2.0 |
| 32.6 | 4.8 |

TABLE 3
¹³C NMR Chemical Shifts from FIG. 1B for Form I.

| | Chemical |
|---|---|
| Peak | Shift (ppm) |
| 1 | 158.3 |
| 2 | 152.8 |
| 3 | 152.2 |
| 4 | 146.0 |
| 5 | 140.6 |
| 6 | 140.1 |
| 7 | 137.8 |
| 8 | 134.6 |
| 9 | 127.9 |
| 10 | 124.0 |
| 11 | 116.8 |
| 12 | 114.6 |
| 13 | 113.6 |
| 14 | 88.7 |
| 15 | 68.8 |
| 16 | 68.3 |
| 17 | 32.1 |
| 18 | 19.9 |

In one embodiment of the invention, Form I of Compound 1 is characterized by the XRPD pattern of FIG. 1A.

In another embodiment of the invention, Form I of Compound 1 has the XRPD characteristics shown in Table 2.

In another embodiment of the invention, Form I of Compound 1 is characterized by at least three XRPD peaks at 2θ angles selected from 12.6°, 14.1°, 14.9°, 21.3°, and 27.1°.

In another embodiment of the invention, Form I of Compound 1 is characterized by XRPD peaks at 2θ angles selected from 12.61°, 14.07°, 14.86°, 21.26°, and 27.1°.

In another embodiment of the invention, Form I of Compound 1 is characterized by XRPD peaks at 2θ angles selected from 6.6°, 12.1°, 12.6°, 14.9°, 17.3°, 19.9°, 20.6°, 21.3°, and 27.1°.

In one embodiment of the invention, Form I of Compound 1 is characterized by the ¹³C solid state NMR spectrum of FIG. 1B.

In another embodiment of the invention, Form I of Compound 1 has the ¹³C solid state NMR characteristics shown in Table 3.

In another embodiment of the invention, Form I of Compound 1 is characterized by ¹³C solid-state nuclear magnetic resonance peaks at chemical shifts selected from 152.8 ppm, 152.2 ppm, 86.7 ppm, 68.8 ppm, and 19.9 ppm.

In another embodiment of the invention, Form I of Compound 1 is characterized by ¹³C solid-state nuclear magnetic resonance peaks at chemical shifts selected from 152.8 ppm, 152.2 ppm, 86.7 ppm, 68.8 ppm, and 19.9 ppm.

In one embodiment of the invention, Form I of Compound 1 is characterized by thermal analysis profiles determined by DSC as shown in FIG. 1C. The DSC analysis of Form I (FIG. 1C) shows a single melt with an onset temperature of 215° C.

In another embodiment of the invention, Form I of Compound 1 is characterized by thermal analysis profiles determined by TGA as shown in FIG. 1D. The TGA analysis of Form I (FIG. 1D) shows a weight loss of 0.02% up to 150° C., consistent with an anhydrous form.

In another embodiment of the invention, Form I of Compound 1 is characterized by DVS isotherms shown in FIGS. 1E-1 and 1E-2. The DVS plot shown in FIG. 1E-2 for Form I shows water uptake of only about 0.4% (wt.) at 95% Rh during the second cycle, which illustrates that the material is non-hygroscopic.

Samples of Form I were maintained for at least 9 months at 25°, and 60% and at least 6 months at 40° and 75% relative humidity. Under both storage conditions, the samples of Form I showed no increase in water content, no increase in impurity levels, no change in particle sized distribution, and no change in solid form was observed

### Characteristics of Form X

The X-ray powder diffraction (XRPD) pattern of Form X of Compound 1 is shown in FIG. 2A; the ¹³C solid state NMR spectrum of Form X of Compound 1 is shown in FIG. 2B; and the thermal analysis profiles of Form X of Compound 1 determined by DSC and TGA measurement are shown in FIGS. 2C and 2D, respectively; and the DVS profile is shown in FIG. 2E.

Characteristic XRPD peaks and ¹³C solid-state nuclear magnetic resonance peaks are provided in Table 4 and Table 5, respectively.

TABLE 4
X-ray powder diffraction (XRPD) characteristics
from FIG. 2A for Form X.

| 2Θ, | Intensity I/I, |
|---|---|
| [°] | [%] |
| 3.9 | 3.4 |
| 4.2 | 5.5 |
| 4.7 | 4.6 |
| 6.6 | 21.8 |
| 9.9 | 3.8 |
| 12.0 | 6.0 |
| 12.9 | 31.8 |
| 13.3 | 9.0 |
| 13.6 | 11.2 |
| 14.5 | 39.5 |
| 15.5 | 57.5 |
| 16.7 | 11.6 |
| 17.2 | 21.9 |
| 17.9 | 70.8 |
| 19.1 | 10.3 |
| 19.6 | 78.5 |
| 20.0 | 50.6 |
| 20.5 | 100.0 |
| 21.8 | 52.8 |
| 22.6 | 6.9 |
| 23.3 | 9.6 |
| 23.9 | 7.5 |
| 24.1 | 6.3 |
| 25.0 | 15.0 |
| 25.3 | 5.4 |
| 26.3 | 19.0 |
| 26.8 | 12.3 |
| 27.8 | 68.0 |
| 28.2 | 12.0 |
| 28.7 | 14.1 |
| 29.4 | 16.5 |
| 30.4 | 9.3 |
| 30.9 | 3.8 |
| 32.7 | 7.6 |
| 33.7 | 3.3 |

TABLE 5
¹³C NMR Chemical Shifts from FIG. 2B for Form X.

| | Chemical |
|---|---|
| Peak | Shift (ppm) |
| 1 | 158.4 |
| 2 | 157.7 |
| 3 | 152.4 |
| 4 | 151.4 |
| 5 | 145.9 |
| 6 | 140.6 |
| 7 | 139.8 |
| 8 | 137.7 |
| 9 | 136.9 |
| 10 | 134.6 |
| 11 | 127.9 |
| 12 | 123.9 |
| 13 | 116.7 |
| 14 | 115.7 |
| 15 | 114.6 |
| 16 | 113.5 |
| 17 | 88.9 |
| 18 | 88.1 |
| 19 | 69.6 |
| 20 | 68.3 |
| 21 | 32.0 |
| 22 | 20.6 |
| 23 | 19.5 |

In one embodiment of the invention, Form X of Compound 1 is characterized by the XRPD pattern of FIG. 2A.

In another embodiment of the invention, Form X of Compound 1 has the XRPD characteristics shown in Table 4.

In another embodiment of the invention, Form X of Compound 1 is characterized by at least five XRPD peaks at 2θ angles selected from 9.9°, 12.9°, 15.5°, 20.5°, 21.8°, 26.3°, and 27.8°.

In another embodiment of the invention, Form X of Compound 1 is characterized by XRPD peaks at 2θ angles selected from 9.9°, 12.9°, 15.9°, 20.5°, 21.8°, 26.3°, and 27.8°.

In another embodiment of the invention, Form X of Compound 1 is characterized by XRPD peaks at 2θ angles selected from 6.6°, 9.29°, 12.88°, 14.54°, 15.49°, 17.88°, 19.63°, 19.95°, 20.47°, 21.84°, 22.25°, and 27.78°.

In one embodiment of the invention, Form X of Compound 1 is characterized by the ¹³C solid state NMR spectrum of FIG. 2B.

In another embodiment of the invention, Form X of Compound 1 has the ¹³C solid state NMR characteristics shown in Table 5.

In another embodiment of the invention, Form X of Compound 1 is characterized by ¹³C solid-state nuclear magnetic resonance peaks at chemical shifts selected from 157.67 ppm, 151.43 ppm, 88.14 ppm, 69.58 ppm, and 20.59 ppm.

In one embodiment of the invention, Form X of Compound 1 is characterized by thermal analysis profiles determined by DSC as shown in FIG. 2C. The DSC analysis of Form X (FIG. 2C) shows a small endotherm with an onset temperature of approximately 204° C. that represents a sublimation/form conversion event before melting as Form I at an onset temperature of about 219° C.

In another embodiment of the invention, Form X of Compound 1 is characterized by thermal analysis profiles determined by TGA as shown in FIG. 2D. The TGA analysis of Form X (FIG. 2D) shows little weight loss (0.2%), confirming that it is a non-solvated form.

In another embodiment of the invention, Form X of Compound 1 is characterized by DVS isotherm shown in FIG. 2E. The DVS plot for Form X shows water uptake of only about 0.07% at 90% Rh during the second cycle, which illustrates that the material is non-hygroscopic.

Samples of Form X were maintained for at least 9 months at 25°, and 60% and at least 6 months at 40° and 75% relative humidity. Under both storage conditions, the samples of Form X showed no increase in water content, no increase in impurity levels, and no change in solid form was observed.

### Characteristics of Form II

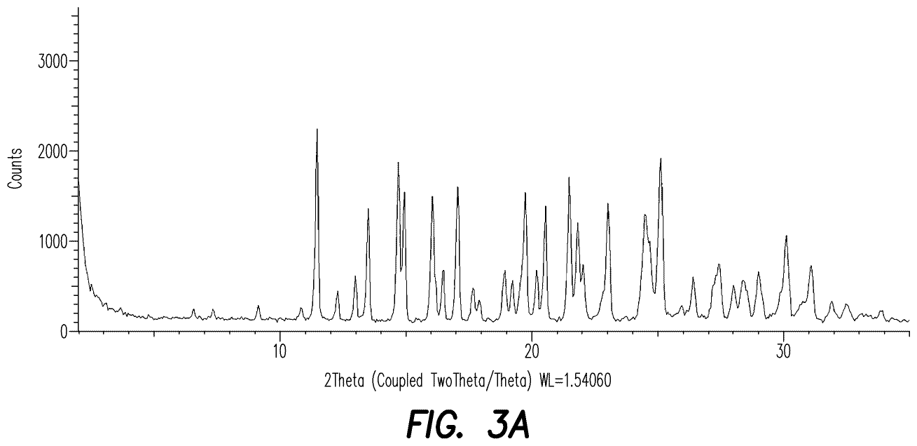

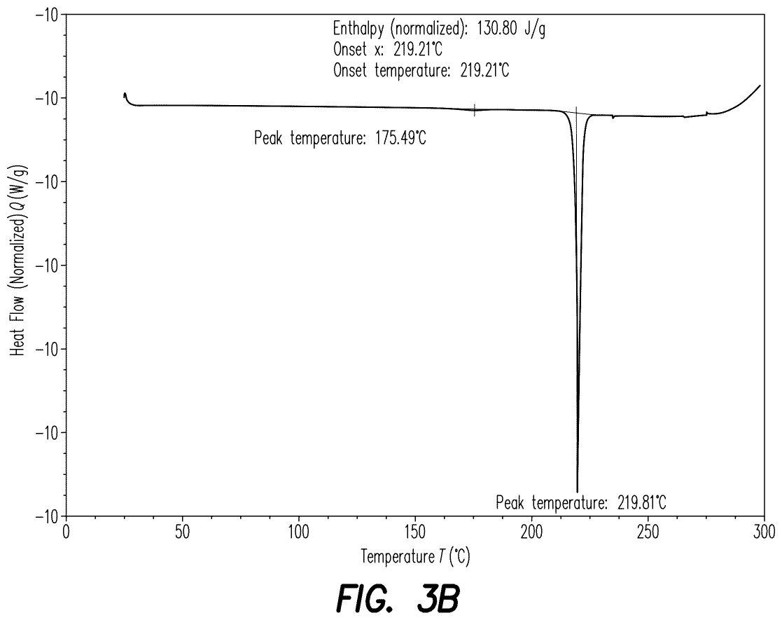

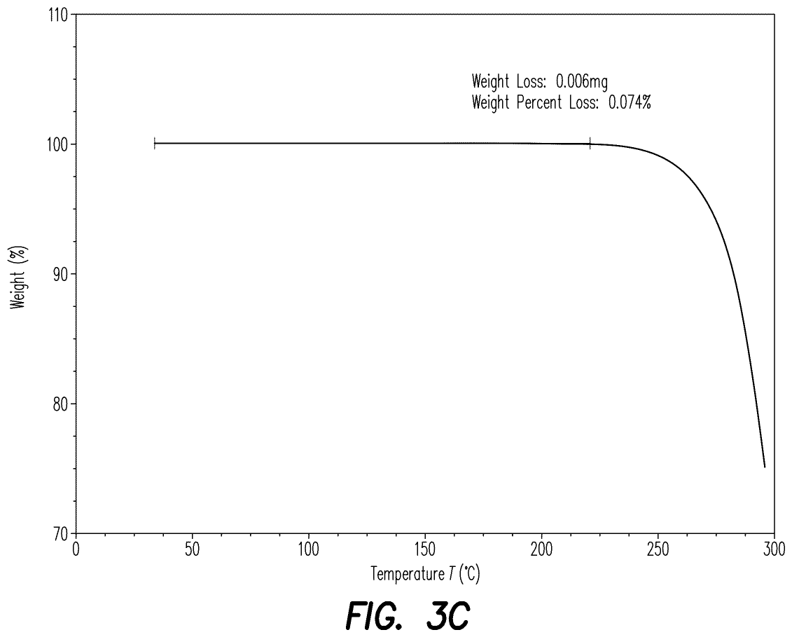

The X-ray powder diffraction (XRPD) pattern of Form II of Compound 1 is shown in FIG. 3A; and the thermal analysis profiles of Form II of Compound 1 determined by DSC and TGA measurement are shown in FIGS. 3B and 3C, respectively. Characteristic XRPD peaks are provided in Table 6.

TABLE 6
X-ray powder diffraction (XRPD) characteristics
from FIG. 3A for Form II.

| 2Θ, | Intensity I/I, |
|---|---|
| [°] | [%] |
| 4.4 | 5.7 |
| 6.5 | 3.5 |
| 7.4 | 5.3 |
| 9.1 | 6.2 |
| 10.9 | 6.3 |
| 11.5 | 100.0 |
| 12.3 | 13.6 |
| 13.0 | 21.0 |
| 13.5 | 57.1 |
| 14.7 | 85.7 |
| 14.9 | 60.6 |
| 16.1 | 65.8 |
| 16.5 | 25.7 |
| 17.1 | 73.6 |
| 17.7 | 16.8 |
| 17.9 | 9.0 |
| 18.9 | 26.8 |
| 19.2 | 19.9 |
| 19.7 | 68.9 |
| 20.5 | 55.6 |
| 21.5 | 77.7 |
| 21.8 | 50.5 |
| 22.1 | 20.1 |
| 23.0 | 63.6 |
| 24.5 | 63.9 |
| 25.1 | 97.5 |
| 25.9 | 7.1 |
| 26.4 | 21.8 |
| 27.4 | 32.0 |
| 28.0 | 16.5 |
| 28.4 | 21.7 |
| 29.0 | 24.6 |
| 30.1 | 46.1 |
| 31.1 | 30.1 |
| 31.9 | 9.3 |
| 32.5 | 8.1 |
| 33.9 | 4.9 |

In one embodiment of the invention, Form II of Compound 1 is characterized by the XRPD pattern of FIG. 3A.

In another embodiment of the invention, Form II of Compound 1 has the XRPD characteristics shown in Table 6.

In another embodiment of the invention, Form II of Compound 1 is characterized by at least five XRPD peaks at 2θ angles selected from 11.46°, 13.49°, 14.70°, 14.92°, 16.05°, 17.06°, 19.72°, 20.52°, 21.43°, 23.03°, 24.51°, 25.11°, and 30.11°.

In another embodiment of the invention, Form II of Compound 1 is characterized by XRPD peaks at 2θ angles selected from 11.46°, 13.49°, 14.70°, 14.92°, 16.05°, 17.06°, 19.72°, 20.52°, 21.43°, 23.03°, 24.51°, 25.11°, and 30.11°.

In one embodiment of the invention, Form II of Compound 1 is characterized by thermal analysis profiles determined by DSC as shown in FIG. 3B. The DSC analysis of Form II (FIG. 3B) shows an onset melting temperature of 219° C., which coincides with the melt of Form I, indicating that there is a conversion from Form I to Form I during heating.

Form II of Compound 1 is characterized by thermal analysis profiles determined by TGA as shown in FIG. 3C. The TGA analysis of Form II (FIG. 3C) shows a weight loss of about 0.1% up to the melt, which is characteristic of a non-solvated form.

### Characteristics of Form III

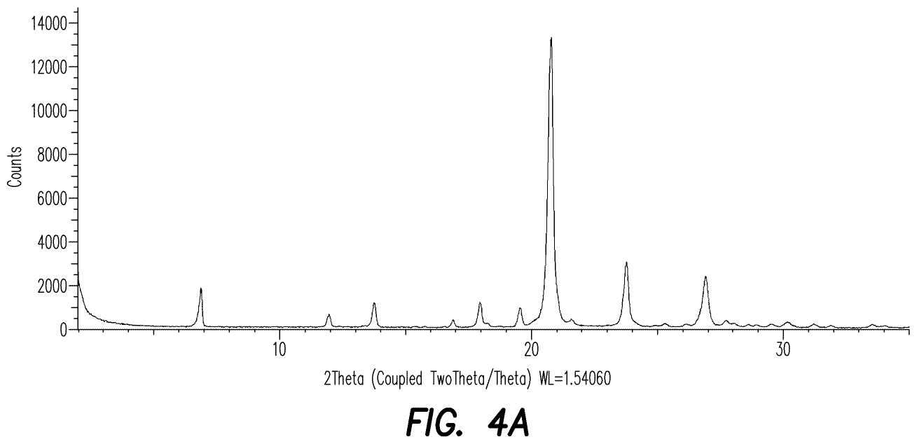

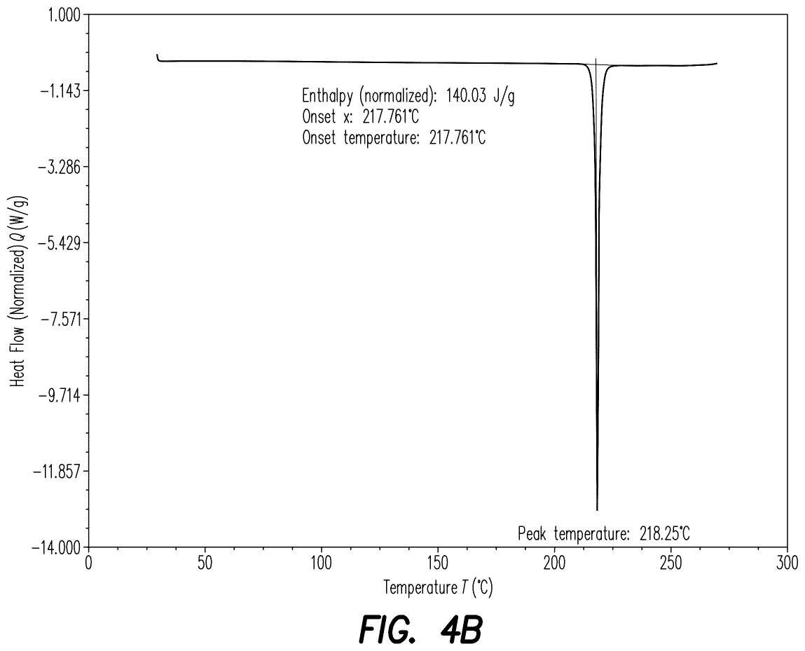

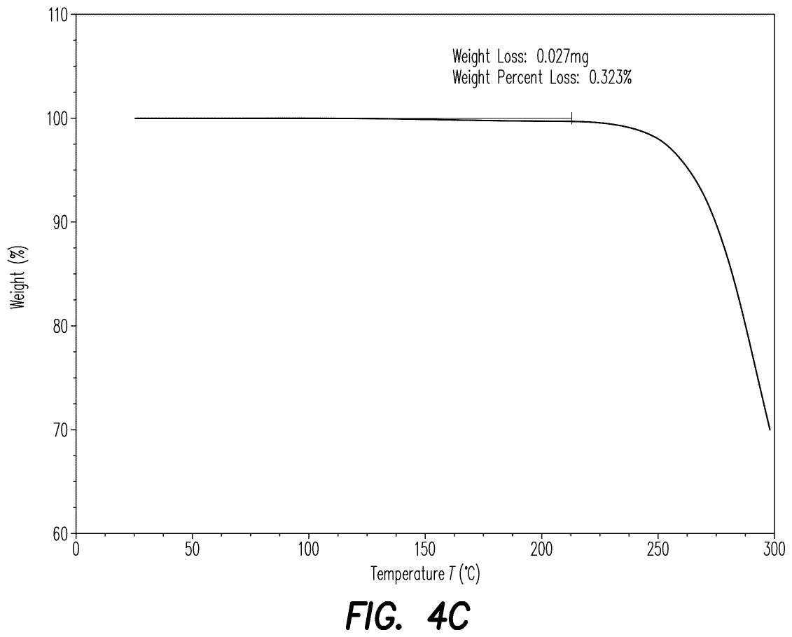

The X-ray powder diffraction (XRPD) pattern of Form III of Compound 1 is shown in FIG. 4A; and the thermal analysis profiles of Form III of Compound 1 determined by DSC and TGA measurement are shown in FIGS. 4B and 4C, respectively. Characteristic XRPD peaks are provided in Table 7.

TABLE 7
X-ray powder diffraction (XRPD) characteristics
from FIG. 4A for Form III.

| 2Θ, | Intensity I/I, |
|---|---|
| [°] | [%] |
| 4.7 | 1.0 |
| 6.8 | 11.5 |
| 11.9 | 3.8 |
| 13.7 | 7.9 |
| 16.9 | 2.0 |
| 17.9 | 7.8 |
| 18.2 | 0.5 |
| 19.5 | 6.0 |
| 20.8 | 100.0 |
| 21.6 | 1.8 |
| 23.8 | 21.1 |
| 25.3 | 0.6 |
| 26.1 | 0.5 |
| 26.9 | 16.8 |
| 27.7 | 1.6 |
| 28.0 | 0.5 |
| 29.5 | 0.4 |
| 30.2 | 1.1 |
| 31.2 | 0.4 |

In one embodiment of the invention, Form III Compound 1 is characterized by the XRPD pattern of FIG. 4A.

In another embodiment of the invention, Form III of Compound 1 has the XRPD characteristics shown in Table 7.

In another embodiment of the invention, Form III of Compound 1 is characterized by at least four XRPD peaks at 2θ angles selected from 6.84°, 13.74°, 17.94°, 20.75°, 23.76°, and 26.91°.

In another embodiment of the invention, Form III of Compound 1 is characterized by XRPD peaks at 2θ angles selected from 6.84°, 13.74°, 17.94°, 20.75°, 23.76°, and 26.91°.

In one embodiment of the invention, Form III of Compound 1 is characterized by thermal analysis profiles determined by DSC as shown in FIG. 4B. The DSC analysis of Form III (FIG. 4B) DSC of Form III shows a small event at about 167° C., which most likely represents a solid-solid conversion to Form I before the Form I melt at about 218° C.

In another embodiment of the invention, Form III of Compound 1 is characterized by thermal analysis profiles determined by TGA as shown in FIG. 4C. The TGA analysis of Form III (FIG. 4C) shows approximately 0.5% weight loss up to the melt, confirming that Form III is a non-solvated form.

### Characteristics of Form IV

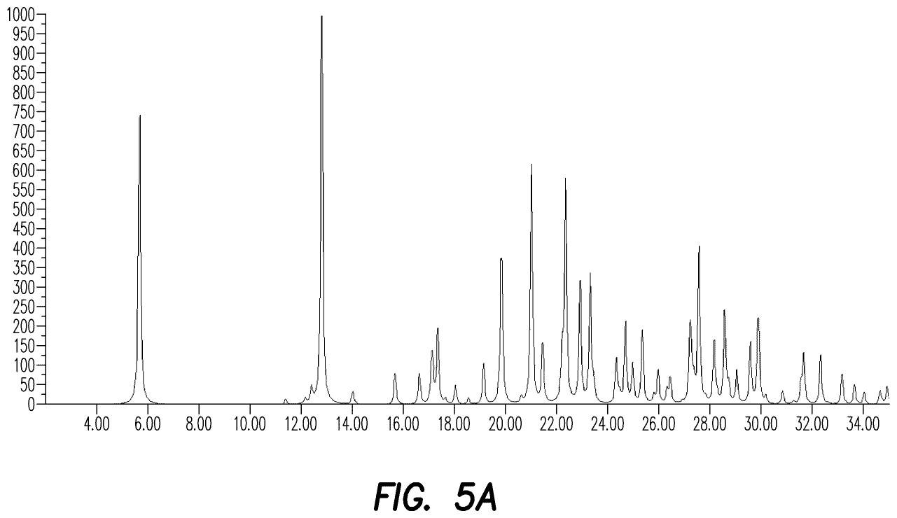

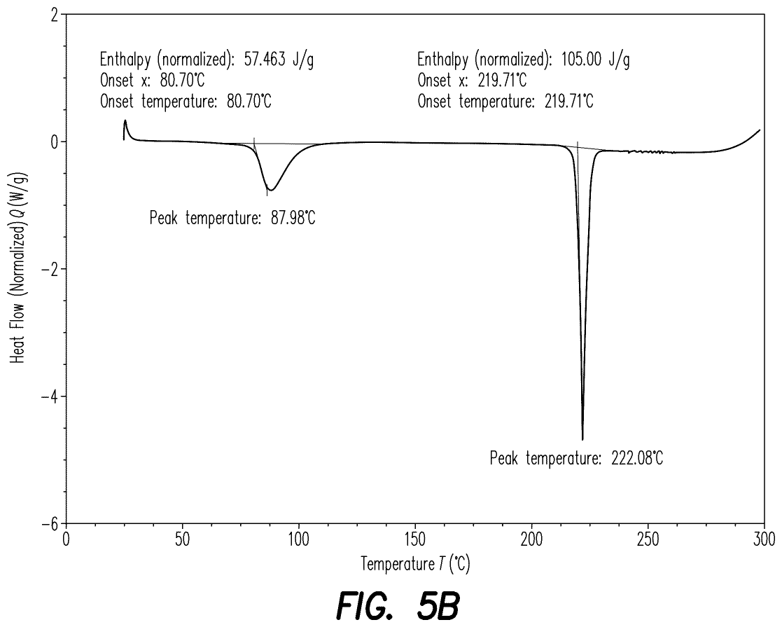

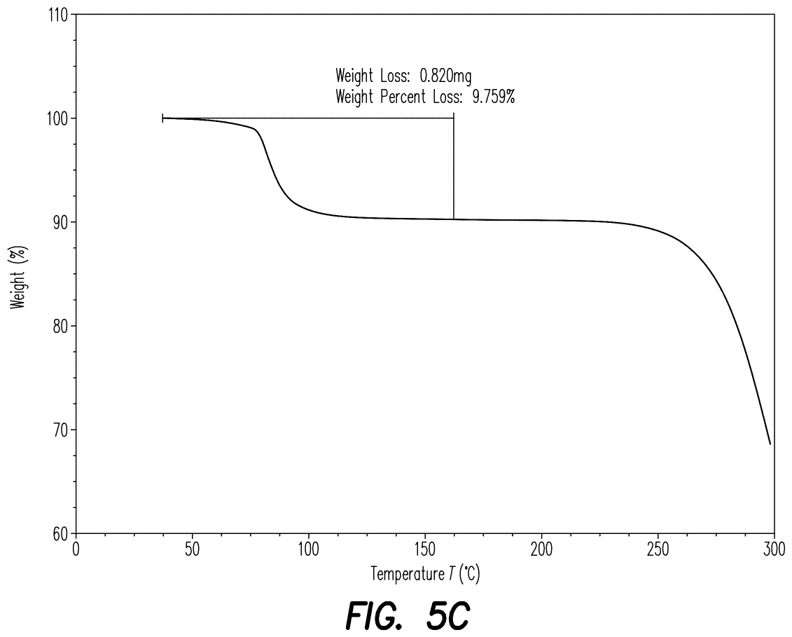

The X-ray powder diffraction (XRPD) pattern of Form IV (acetonitrile solvate of Compound 1) is shown in FIG. 5A; and the thermal analysis profiles of Form IV of Compound 1 determined by DSC and TGA measurement are shown in FIGS. 5B and 5C, respectively. Characteristic XRPD peaks are provided in Table 8.

TABLE 8
X-ray powder diffraction (XRPD) characteristics
from FIG. 5A for Form IV.

| 2Θ, | Intensity I/I, |
|---|---|
| [°] | [%] |
| 5.7 | 73.9 |
| 12.8 | 100.0 |
| 14.1 | 2.9 |
| 15.7 | 7.1 |
| 16.7 | 7.1 |
| 17.4 | 18.1 |
| 18.1 | 4.2 |
| 19.2 | 9.3 |
| 19.9 | 35.4 |
| 21.0 | 59.7 |
| 21.5 | 14.2 |
| 22.4 | 56.1 |
| 22.9 | 29.3 |
| 23.3 | 30.9 |
| 24.4 | 10.9 |
| 24.7 | 19.3 |
| 25.0 | 9.1 |
| 25.4 | 17.1 |
| 26.0 | 7.7 |
| 26.5 | 6.0 |
| 27.3 | 21.4 |
| 27.6 | 37.6 |
| 28.2 | 14.9 |
| 28.6 | 22.0 |
| 29.1 | 7.5 |
| 29.6 | 14.4 |
| 29.9 | 21.4 |
| 30.9 | 2.5 |
| 31.7 | 11.7 |
| 32.4 | 11.0 |
| 33.2 | 6.3 |
| 33.7 | 3.8 |
| 34.0 | 1.9 |
| 34.7 | 2.0 |
| 34.9 | 2.8 |

In one embodiment of the invention, Form IV Compound 1 is characterized by the XRPD pattern of FIG. 5A.

In another embodiment of the invention, Form IV of Compound 1 has the XRPD characteristics shown in Table 8.

In another embodiment of the invention, Form IV of Compound 1 is characterized by at least five XRPD peaks at 2θ angles selected from 5.71°, 12.83°, 19.85°, 21.03°, 22.35°, 22.91°, 23.31°, and 27.59°.

In another embodiment of the invention, Form IV of Compound 1 is characterized by XRPD peaks at 2θ angles selected from 5.71°, 12.83°, 19.85°, 21.03°, 22.35°, 22.91°, 23.31°, and 27.59°.

In one embodiment of the invention, Form IV of Compound 1 is characterized by thermal analysis profiles determined by DSC as shown in FIG. 5B. The DSC analysis of Form IV (FIG. 5B) shows an initial endotherm with an onset of 78° C. which most likely corresponds to loss of solvent, followed by a melt with an onset temperature of about 216° C. which corresponds to melting of Form 1.

In another embodiment of the invention, Form IV of Compound 1 is characterized by thermal analysis profiles determined by TGA as shown in FIG. 5C. The TGA analysis of Form IV (FIG. 5C) shows approximately 8.0% weight loss from 40° C. to 210°, which is attributed to a loss of 0.7 acetonitrile molecules per molecule (Compound 1).

### Characteristics of Form VI

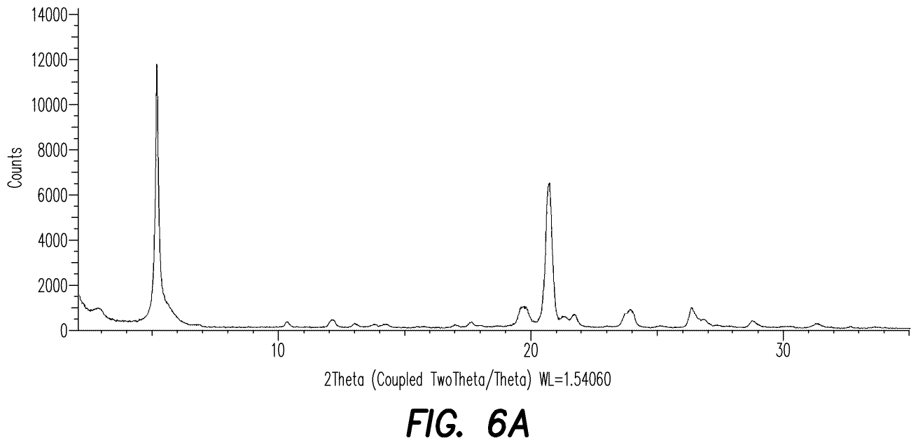

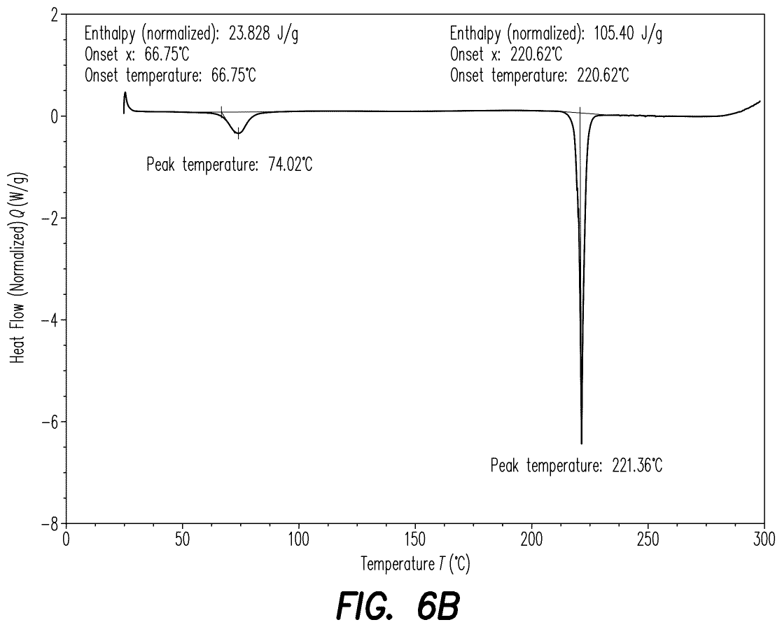

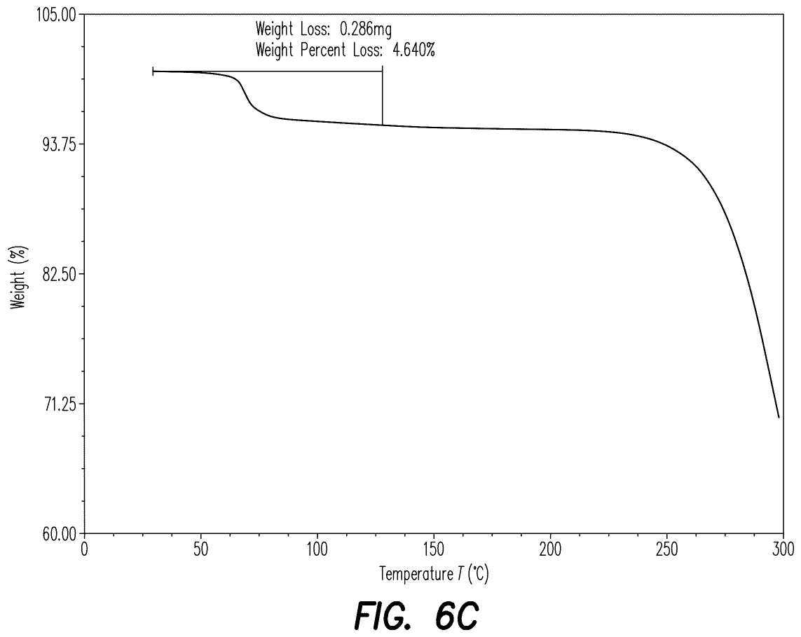

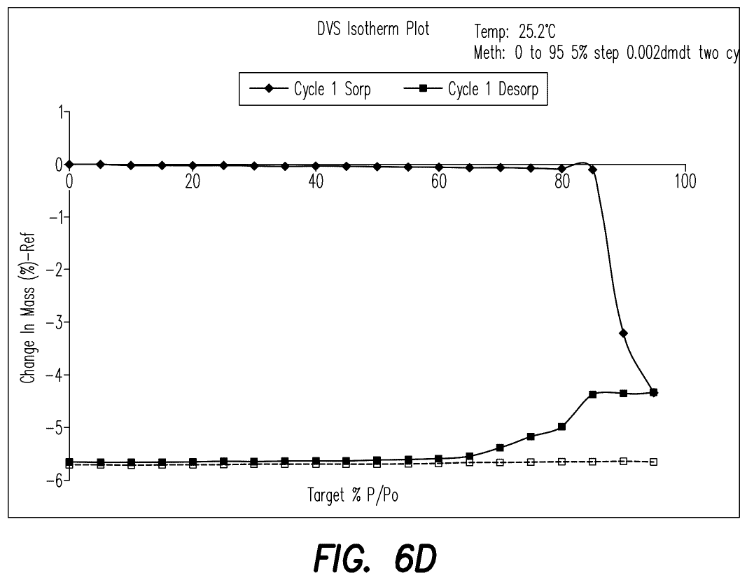

The X-ray powder diffraction (XRPD) pattern of Form VI (acetone solvate of Compound 1) is shown in FIG. 6A; and the thermal analysis profiles of Form VI of Compound 1 determined by DSC and TGA measurement are shown in FIGS. 6B and 6C, respectively, and the DVS profile is shown in FIG. 6D.

Characteristic XRPD peaks are provided in Table 9.

TABLE 9
X-ray powder diffraction (XRPD) characteristics
from FIG. 6A for Form IV.

| 2Θ, | Intensity I/I, |
|---|---|
| [°] | [%] |
| 4.0 | 1.0 |
| 5.2 | 100.0 |
| 6.8 | 0.6 |
| 10.3 | 2.1 |
| 12.1 | 2.9 |
| 13.0 | 1.4 |
| 13.8 | 1.2 |
| 14.2 | 1.2 |
| 17.0 | 0.9 |
| 17.7 | 1.9 |
| 18.0 | 0.6 |
| 18.6 | 0.4 |
| 19.7 | 8.1 |
| 20.7 | 58.7 |
| 21.3 | 2.8 |
| 21.7 | 2.8 |
| 24.0 | 7.0 |
| 26.4 | 7.8 |
| 26.8 | 2.3 |
| 27.4 | 0.5 |
| 28.7 | 2.3 |
| 31.3 | 1.2 |

In one embodiment of the invention, Form VI Compound 1 is characterized by the XRPD pattern of FIG. 6A.

In another embodiment of the invention, Form VI of Compound 1 has the XRPD characteristics shown in Table 9.

In another embodiment of the invention, Form VI of Compound 1 is characterized by at least five XRPD peaks at 2θ angles selected from 5.2°, 12.1° C., 19.7°, 20.7°, 24.0°, and 26.4°.

In another embodiment of the invention, Form VI of Compound 1 is characterized by XRPD peaks at 2θ angles selected from 5.2°, 12.1° C., 19.7°, 20.7°, 24.0°, and 26.4°. In another embodiment of the invention, Form VI of Compound 1 is characterized by thermal analysis profiles determined by DSC as shown in FIG. 6B. The DSC analysis of Form VI (FIG. 6B) shows an initial endotherm at 69° C., which corresponds to loss of acetone, followed by a melting endotherm at 218° C. which is attributed to melting of Form I.

In another embodiment of the invention, Form VI of Compound 1 is characterized by thermal analysis profiles determined by TGA as shown in FIG. 6C. The TGA analysis of Form VI (FIG. 6C) shows a weight loss of about 11%, slightly lower than the expected loss of 15% which would account for a theoretical monosolvate.

The DVS profile (FIG. 6D) data shows that the mass of form VI was stable over a wide humidity range from RH=0 to the 80%. A sharp descending step occurred when the 80% RH was reached. Based on further DVS cycles of the resulting phase, acetone solvent molecules are replaced by water molecules and the acetone solvate is transformed to a mixture of Form I and Form III.

### Characteristics of Form VII

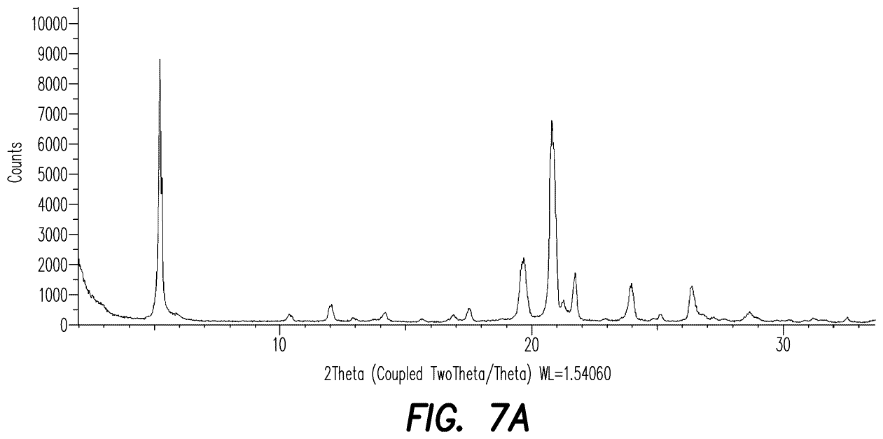

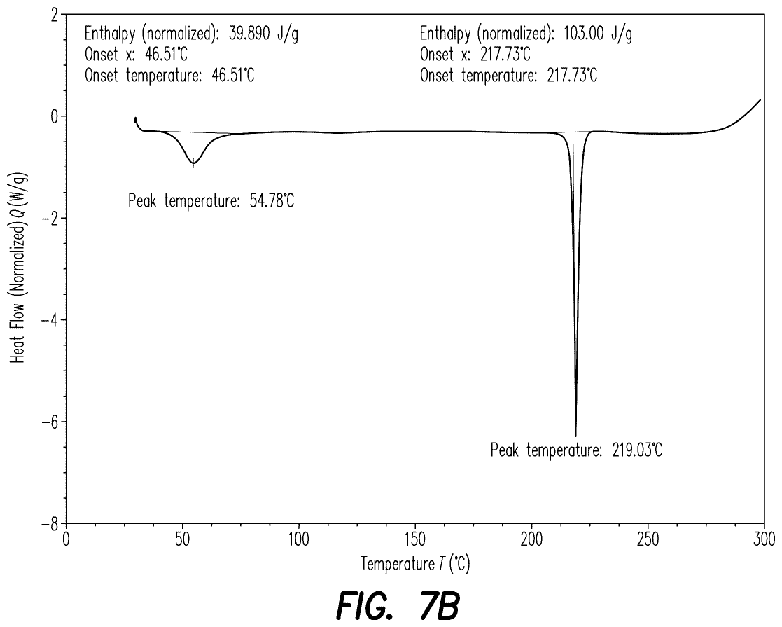

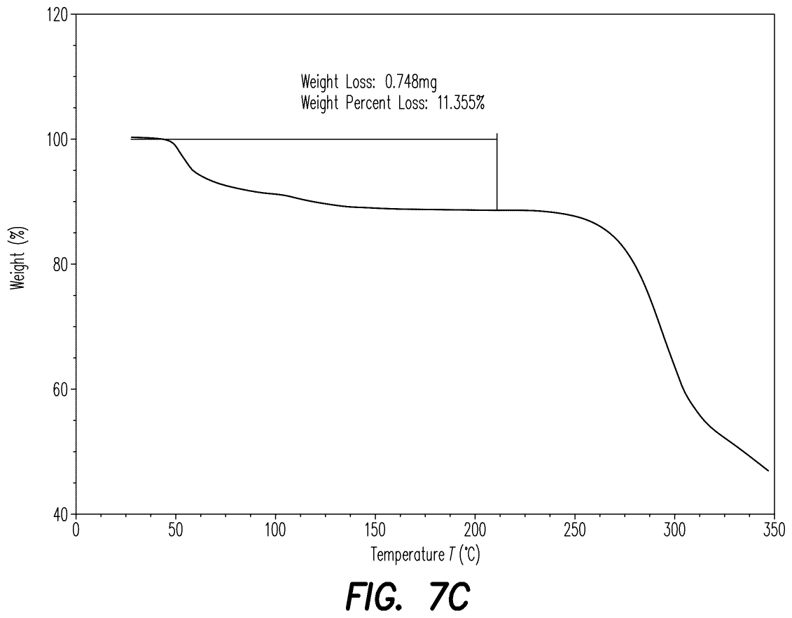

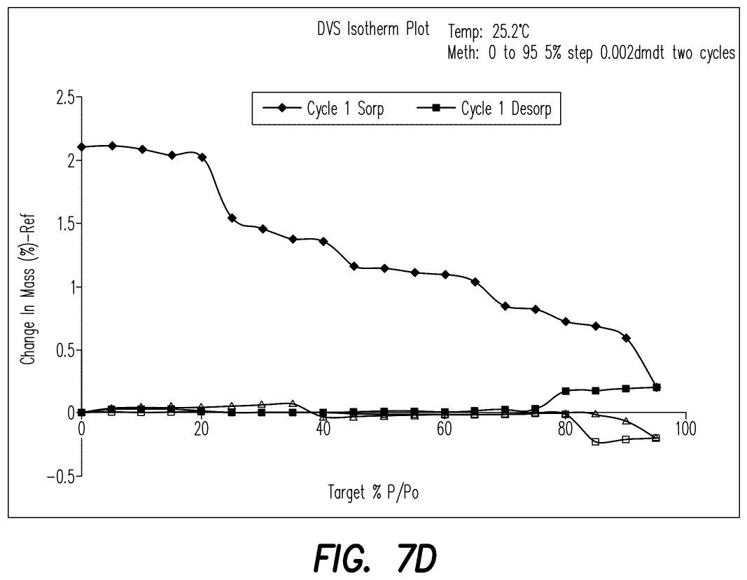

The X-ray powder diffraction (XRPD) pattern of Form VII (THF solvate of Compound 1) is shown in FIG. 7A; and the thermal analysis profiles of Form VII of Compound 1 determined by DSC and TGA measurement are shown in FIGS. 7B and 7C, respectively, and the DVS profile is shown in FIG. 7D.

Characteristic XRPD peaks are provided in Table 10.

TABLE 10
X-ray powder diffraction (XRPD) characteristics
from FIG. 7A for Form VII.

| 2Θ, | Intensity I/I, |
|---|---|
| [°] | [%] |
| 3.9 | 1.3 |
| 4.0 | 1.6 |
| 4.2 | 1.6 |
| 5.2 | 100.0 |
| 5.8 | 1.5 |
| 10.4 | 2.7 |
| 12.0 | 6.4 |
| 12.9 | 1.5 |
| 13.7 | 0.9 |
| 14.2 | 3.7 |
| 15.7 | 1.1 |
| 16.9 | 2.6 |
| 17.5 | 5.1 |
| 18.8 | 1.1 |
| 19.7 | 25.3 |
| 20.8 | 87.1 |
| 21.2 | 5.7 |
| 21.7 | 18.3 |
| 22.9 | 1.0 |
| 24.0 | 14.6 |
| 25.1 | 2.7 |
| 26.4 | 14.3 |
| 26.8 | 2.3 |
| 27.3 | 1.3 |
| 28.7 | 3.5 |
| 31.2 | 1.3 |
| 32.5 | 1.4 |
| 33.8 | 1.2 |

In one embodiment of the invention, Form IVI Compound 1 is characterized by the XRPD pattern of FIG. 7A.

In another embodiment of the invention, Form VII of Compound 1 has the XRPD characteristics shown in Table 10.

In another embodiment of the invention, Form VII of Compound 1 is characterized by at least five XRPD peaks at 2θ angles selected from 5.2°, 19.66°, 20.8°, 21.72°, 23.98°, and 26.36°.

In another embodiment of the invention, Form VII of Compound 1 is characterized by XRPD peaks at 2θ angles selected from 5.2°, 19.66°, 20.8°, 21.72°, 23.98°, and 26.36 In another embodiment of the invention, Form VII of Compound 1 is characterized by thermal analysis profiles determined by DSC as shown in FIG. 7B. The DSC analysis of Form VII (FIG. 7B) shows an initial endotherm with an onset of approximately 51° C., which corresponds to loss of THF, followed by a melting endotherm with an onset of about 218° C., which is attributed to melting of Form 1.

In another embodiment of the invention, Form VII of Compound 1 is characterized by thermal analysis profiles determined by TGA as shown in FIG. 7C. The TGA analysis of Form VII (FIG. 7C) shows a weight loss of about 16%, which is consistent with a theoretical monosolvate (18% loss).

DVS data (FIG. 7D) shows the occurrence of a solvent replacement, starting from low humidity. THF solvent molecules are replaced by water molecules, and the THF solvate is transformed to a mixture of Form I and Form III.

### Characteristics of Form IX

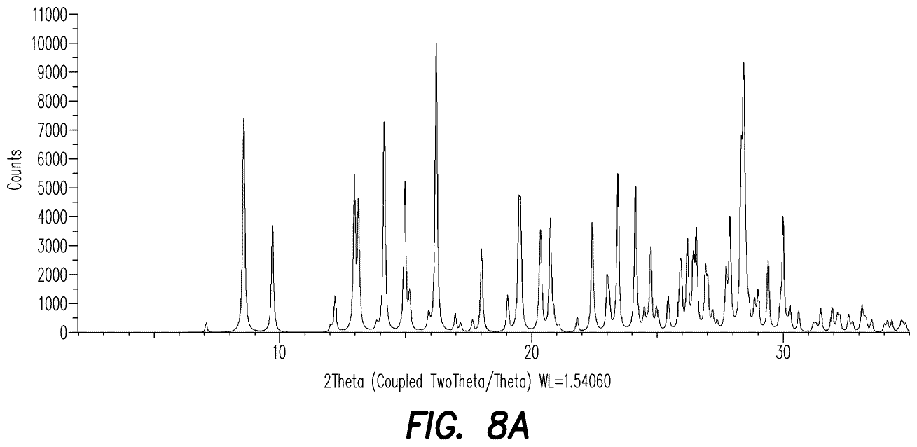

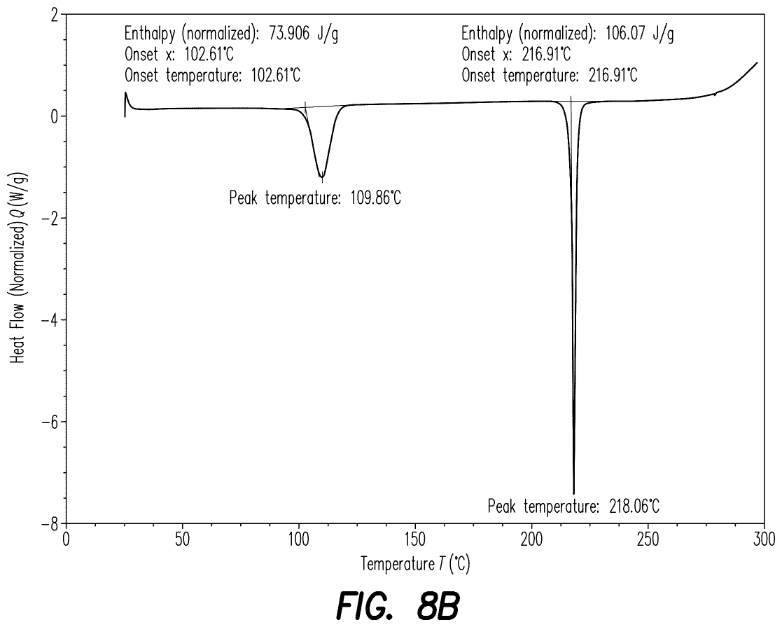

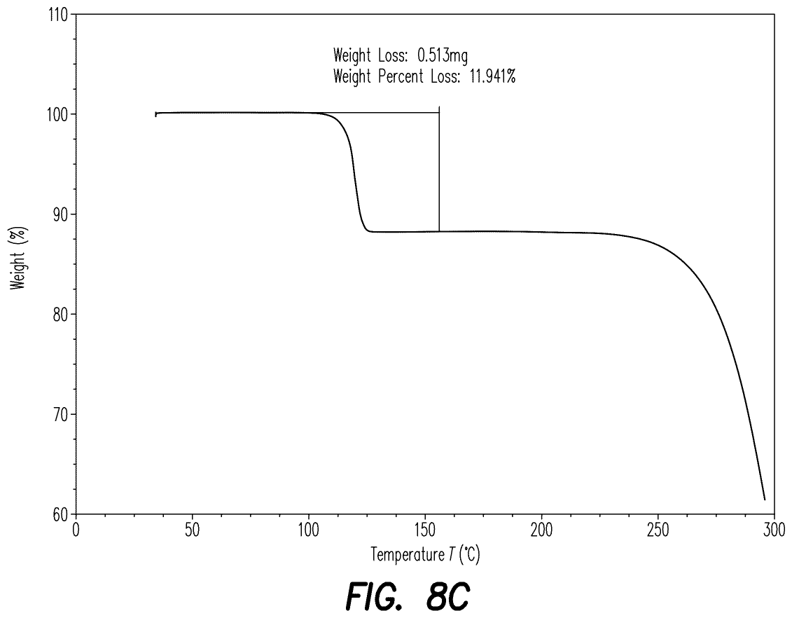

The X-ray powder diffraction (XRPD) pattern of Form IX (toluene solvate of Compound 1) is shown in FIG. 8A; and the thermal analysis profiles of Form IX of Compound 1 determined by DSC and TGA measurement are shown in FIGS. 8B and 8C, respectively. Characteristic XRPD peaks are provided in Table 11.

TABLE 11
X-ray powder diffraction (XRPD) characteristics
from FIG. 8A for Form IX.

| 2Θ, | Intensity I/I, |
|---|---|
| [°] | [%] |
| 7.1 | 3.1 |
| 8.6 | 72.4 |
| 9.7 | 34.7 |
| 12.2 | 11.6 |
| 13.0 | 52.8 |
| 13.1 | 39.5 |
| 14.1 | 70.5 |
| 15.0 | 49.6 |
| 15.1 | 11.1 |
| 16.2 | 100.0 |
| 17.0 | 5.8 |
| 17.7 | 4.0 |
| 18.0 | 26.5 |
| 19.0 | 11.5 |
| 19.5 | 52.8 |
| 20.4 | 34.1 |
| 20.7 | 36.6 |
| 21.8 | 4.3 |
| 22.4 | 35.1 |
| 23.1 | 19.4 |
| 23.5 | 52.9 |
| 24.2 | 47.4 |
| 24.8 | 27.3 |
| 25.0 | 6.7 |
| 25.5 | 11.0 |
| 25.9 | 26.0 |
| 26.2 | 29.0 |
| 26.5 | 33.7 |
| 26.9 | 23.0 |
| 27.2 | 6.4 |
| 27.4 | 3.3 |
| 27.9 | 37.5 |
| 28.4 | 97.3 |
| 29.0 | 12.9 |
| 29.4 | 22.3 |
| 30.0 | 37.2 |
| 30.3 | 7.6 |
| 30.6 | 6.0 |
| 31.5 | 7.0 |
| 31.9 | 7.6 |
| 32.2 | 5.9 |
| 32.6 | 5.3 |
| 33.1 | 8.5 |
| 33.5 | 3.3 |
| 34.1 | 3.2 |
| 34.3 | 3.2 |
| 34.7 | 3.7 |
| 35.7 | 3.6 |
| 36.3 | 3.6 |
| 36.5 | 2.2 |
| 37.2 | 4.5 |
| 38.6 | 5.0 |
| 39.5 | 5.4 |
| 40.3 | 9.5 |
| 40.8 | 5.9 |
| 41.3 | 5.3 |
| 41.6 | 5.1 |
| 44.1 | 5.4 |
| 44.9 | 3.8 |
| 46.0 | 3.1 |
| 47.1 | 3.4 |
| 47.5 | 5.3 |
| 48.2 | 7.7 |
| 48.5 | 4.1 |

In one embodiment of the invention, Form IX Compound 1 is characterized by the XRPD pattern of FIG. 8A.

In another embodiment of the invention, Form IX of Compound 1 has the XRPD characteristics shown in Table 11.

In another embodiment of the invention, Form IX of Compound 1 is characterized by at least five XRPD peaks at 2θ angles selected from 8.56°, 9.7°, 12.96°, 13.12°, 14.14°, 14.96°, 16.22°, 19.52°, 20.36°, 20.74°, 23.46°, 24.16°, 28.42°, and 30.0°.

In another embodiment of the invention, Form IX of Compound 1 is characterized by XRPD peaks at 2θ angles selected from 8.56°, 9.7°, 12.96°, 13.12°, 14.14°, 14.96°, 16.22°, 19.52°, 20.36°, 20.74°, 23.46°, 24.16°, 28.42°, and 30.0°.

In another embodiment of the invention, Form IX of Compound 1 is characterized by thermal analysis profiles determined by DSC as shown in FIG. 8B. The DSC analysis of Form VII (FIG. 8B) shows an initial endotherm with an onset temperature of approximately 103° C., corresponding to loss of toluene from the crystal lattice, followed by a melting endotherm with an onset of about 217° C., which is attributed to the melt of Form I.

In another embodiment of the invention, Form IX of Compound 1 is characterized by thermal analysis profiles determined by TGA as shown in FIG. 8C. The TGA analysis of Form VII (FIG. 8C) shows a weight loss of about 12% which is consistent with the loss expected from a theoretical hemi toluene solvate

As shown above, Forms I and X are more stable (for example, more resistant to change in form) than Form II, Form III and the crystalline solvate forms of Compound 1. This suggests that Form I and Form X are more suitable for use in a solid form pharmaceutical preparation. Despite the lower stability of the other crystalline forms of Compound 1 (Forms II, Ill, IV, VI, VII and IX), the other crystalline forms are still useful as intermediates to prepare Form I and/or Form X as described herein.

### Methods of Preparing the Compounds of the Invention

Specific conditions for the preparation of the solid forms of Compound 1 of the invention are described in the Examples. The compounds of the invention may be prepared by the general process described below:
(i) Dissolving Compound 1 in a suitable solvent (“the dissolution step”) at elevated temperature,
(ii) Optionally, filtering the solution of step (i) to provide a filtered solution of Compound 1 (“the filtered first solution”), and
(iii) Concentrating and/or cooling the filtered solution of step (ii) to provide a mixture, and
(iv) Isolating the solids from the mixture of step (iii) to provide the solid form of Compound 1.

The desired solid form of Compound 1 is prepared by the judicious choice of suitable solvent in step (i). Suitable solvents comprising acetonitrile, acetone, tetrahydrofuran or toluene can be used to prepare the solvated forms of Compound 1, that is, Forms IV, VI, VII or IX.

In general, any form of Compound 1 may be used in step (i).

In some embodiments, step (ii) is carried out at elevated temperature.

In some embodiments, an antisolvent is added to the filtered solution from step (ii) (“forward addition”). In some embodiments, the filtered solution from step (ii) is added to an antisolvent (“reverse addition”). Nonlimiting examples of antisolvents include water, heptane, chloroform, tert-Butyl methyl ether, cumene, di-isopropyl ether, diethyl ether, cyclopentyl methyl ether, 2-propanol, and 2-butanol.

In some embodiments, a seed crystal is added to the filtered solution from step (ii) or during the concentration and/or cooling of step (iii).

### Methods for Preparing Form I

In one embodiment, the invention relates to a method of making Form I as described in the general procedure above, wherein step (i) is carried out in a mixture of THF (75%) and water (25%).

In one embodiment, the invention relates to a method of making Form I as described in the general procedure above, wherein step (i) is carried out in a mixture of acetone (85%) and water (15%).

In one embodiment, the invention relates to a method of making Form I, wherein the mixture from step (iii) is reheated and cooled at least once prior to isolating Form I in step (iv) (“the heat cycle”). In another embodiment, the heat cycle is carried out twice prior to isolating Form I in step (iv).

In one embodiment, the invention relates to a method of making Form I, wherein a seed crystal of Form I is added to the filtered solution from step (ii) or during the concentration and/or cooling of step (iii).

### Methods for Preparing Form X

In another embodiment, the invention relates to a method of making Form X as described in the general procedure, wherein
step (i) is carried out in a mixture of acetone (85%) and water (15%), and
acetone is added to the cooled slurry comprising Form X after step (iii) to provide a slurry with a solvent composition 50/50 acetone/H₂O by weight, and
wet milling the slurry for sufficient time to convert the substantially all solids to Form X prior to isolating Form X.

In another embodiment, the invention relates to a method for making Form X, as described immediately above, wherein a seed crystal of Form X is added to the filtered solution from step (ii) or during the concentration and/or cooling of step (iii).

In another embodiment, the invention relates to a method of making Form X, comprising:
(i) Dissolving Compound 1 in nitromethane at elevated temperature,
(ii) Optionally, filtering the solution of step (i) to provide a filtered solution of Compound 1 (“the filtered first solution”), and
(iii) Concentrating and/or cooling the filtered solution of step (ii) to provide a mixture,
(iv) Isolating the solids from the mixture of step (iii) to provide a nitromethane solvate of Compound 1,
(v) Treating the nitromethane solvate from step (iv) with MIBK to provide a slurry,
(vi) Mixing the slurry at room temperature and for sufficient time to convert the solids to Form X,
(vii) Isolating the solids from the slurry of step (vii) to provide Form X.

### Methods for Preparing Form II

In one embodiment, the invention relates to a method of making Form II as described in the general procedure above, wherein step (i) is carried out in a mixture of DMSO and water.

In another embodiment, the invention relates to a method of making Form II as described in the general procedure above, wherein step (i) is carried out in methanol.

### Methods for Preparing Form III

In one embodiment, the invention relates to a method of making Form III as described in the general procedure above, wherein step (i) is carried out in a mixture of methyl isobutyl ketone and ketone.

In another embodiment, the invention relates to a method of making Form III as described in the general procedure above, wherein step (i) is carried out in methyl isobutyl ketone, and the solution from step (iii) is rapidly cooled to 4° C.

### Methods for Preparing Form IV

In one embodiment, the invention relates to a method of making Form IV as described in the general procedure above, wherein step (i) is carried out in acetonitrile.

### Methods for Preparing Form VI

In one embodiment, the invention relates to a method of making Form VI as described in the general procedure above, wherein step (i) is carried out in acetone.

### Methods for Preparing Form VII

In one embodiment, the invention relates to a method of making Form VII as described in the general procedure above, wherein step (i) is carried out in THF.

### Methods for Preparing Form IX

In one embodiment, the invention relates to a method of making Form IX as described in the general procedure above, wherein step (i) is carried out in toluene.

In another embodiment, the invention relates to a method of making Form IX, comprising:
(i) Mixing a slurry comprising the amorphous form of Compound 1 with toluene at room temperature and for sufficient time to convert the solids to Form IX, and
(ii) Isolating the solids from the slurry of step (i) to provide Form IX.

### Methods of Therapeutic Use

The compounds disclosed herein effectively inhibit aldosterone synthase. The inhibition of aldosterone synthase is an attractive means for preventing and treating certain diseases and disorders. Nonlimiting examples of such diseases or disorders include those described herein and in WO 2016/014736 and WO202114170.

In one embodiment, the present invention relates to methods for treating, preventing, or slowing the progression of diseases or conditions which can be influenced by inhibition of AS, comprising administering a pharmaceutically effective amount of a compound of the invention to a patient in need thereof.

In another embodiment, the present invention relates to methods for treating, preventing, or slowing the progression of chronic kidney disease, diabetic kidney disease, heart failure including heart failure with reduced ejection fraction (HFrEF), heart failure with preserved ejection fraction (HFpEF), heart failure with LVEF≥40%, heart failure with LVEF<40%, and/or resistant hypertension (rHPT), comprising administering a pharmaceutically effective amount of a compound of the invention to a patient in need thereof. In one embodiment, the compound of the invention is Form I; in another embodiment, the compound of the invention is Form X; in another embodiment, the compound of the invention is a mixture of Form I and Form X.

In one embodiment, the invention relates to the use of a compound of the invention for treating, preventing, or slowing the progression of chronic kidney disease, diabetic kidney disease, heart failure including heart failure with reduced ejection fraction (HFrEF), heart failure with preserved ejection fraction (HFpEF) heart failure with LVEF≥40%, heart failure with LVEF<40%, and/or resistant hypertension (rHPT). The use comprises the manufacture of medicaments for the treatment of the corresponding diseases or disorders described herein. In one embodiment, the compound of the invention is Form I; in another embodiment, the compound of the invention is Form X; in another embodiment, the compound of the invention is a mixture of Form I and Form X.

In one embodiment, the method or use of the invention is for treating, preventing, or slowing the progression of chronic kidney disease.

In another embodiment, the method or use of the invention is for treating, preventing, or slowing the progression of diabetic kidney disease.

In another embodiment, the method or use of the invention is for treating, preventing, or slowing the progression of heart failure.

In another embodiment, the method or use of the invention is for treating, preventing, or slowing the progression of heart failure with reduced ejection fraction (HFrEF).

In another embodiment, the method or use of the invention is for treating, preventing, or slowing the progression of heart failure with preserved ejection fraction (HFpEF).

In another embodiment, the method or use of the invention is for treating, preventing, or slowing the progression of heart failure with LVEF≥40%.

In another embodiment, the method or use of the invention is for treating, preventing, or slowing the progression of heart failure with LVEF≤40%,

In another embodiment, the method or use of the invention is for treating, preventing, or slowing the progression of resistant hypertension (rHPT).

According to one embodiment, the method or use of the invention relates for treating, preventing, or slowing the progression of chronic kidney disease, diabetic kidney disease, heart failure including heart failure with reduced ejection fraction (HFrEF), heart failure with preserved ejection fraction (HFpEF), heart failure with LVEF≥40%, heart failure with LVEF<40%, and/or resistant hypertension (rHPT) in a patient in need thereof, characterized in that a pharmaceutical composition or pharmaceutical dosage form as defined hereinbefore and hereinafter is administered to the patient.

In another embodiment, the invention relates to any of the methods or uses described above, further comprising administering the compound of the invention in combination with an SGLT2 inhibitor. Examples of SGLT2 inhibitors include bexaglifloxin, canagliflozin, dapagliflozin, empagliflozin and ertugliflozin. In one embodiment, the SGLT2 inhibitor in the methods of uses of the invention is empagliflozin.

For therapeutic use, the compounds of the invention may be administered via a pharmaceutical composition in any conventional solid pharmaceutical dosage form in any conventional manner. Conventional solid dosage forms typically include a pharmaceutically acceptable carrier suitable to the particular dosage form selected. Routes of administration include, but are not limited to, orally or by inhalation. The preferred mode of administration is oral.

Preferred doses of the compound of the invention for oral administration are 0.1 to 100 mg; or 1 to 50 mg; or 1 to 25 mg; or 1 to 20 mg. In another embodiment, the preferred dose of the compound of the invention for oral administration is selected from 1 mg, 1.5 mg, 2 mg, 2.5 mg, 3 mg, 3.5 mg, 4, mg, 4.5 mg, 5 mg, 5.5 mg, 6 mg, 6.5 mg, 7 mg, 7.5 mg, 8 mg, 8.5 mg, 9 mg, 9.5 mg, and 10 mg, 10.5 mg, 11 mg, 12 mg, 13 mg, 14 mg, 15 mg, 16 mg, 17 mg, 18 mg, 19 mg, 20 mg, 21 mg, 22 mg, 23 mg, 24 mg and 25 mg.

In one embodiment, the compound of the invention may be administered once per day, twice per day, or three or more times per day. In another embodiment, the compound of the invention may be administered once per week, twice per week, or three or more times per week.

In another embodiment, the compound of the invention is administered in a daily amount of 3 mg, 10 mg, or 20 mg.

In another embodiment, the compound of the invention is administered to the patient once daily in an amount of 3 mg, or 10 mg, or 20 mg.

The compounds of this invention may be administered alone or in combination with adjuvants that enhance stability of the inhibitors, facilitate administration of pharmaceutical compositions containing them in certain embodiments, provide increased dissolution or dispersion, increase inhibitory activity, provide adjunct therapy, and the like, including other active ingredients. In one embodiment, for example, multiple compounds of the present invention can be administered.

Advantageously, such combination therapies utilize lower dosages of the conventional therapeutics, thus avoiding possible toxicity and adverse side effects incurred when those agents are used as monotherapies. Compounds of the invention may be physically combined with the conventional therapeutics or other adjuvants into a single pharmaceutical composition. Advantageously, the compounds may then be administered together in a single dosage form. In some embodiments, the pharmaceutical compositions comprising such combinations of compounds contain at least about 5%, but more preferably at least about 20%, of a compound of formula (I) (w/w) or a combination thereof. The optimum percentage (w/w) of a compound of the invention may vary and is within the purview of those skilled in the art. Alternatively, the compounds of the present invention and the conventional therapeutics or other adjuvants may be administered separately (either serially or in parallel). Separate dosing allows for greater flexibility in the dosing regimen.

As mentioned above, dosage forms of the compounds of this invention may include pharmaceutically acceptable carriers and adjuvants known to those of ordinary skill in the art and suitable to the dosage form. These carriers and adjuvants include, for example, ion exchangers, alumina, aluminum stearate, lecithin, serum proteins, buffer substances, water, salts or electrolytes and cellulose-based substances. Preferred dosage forms include tablet, capsule, caplet, and granule. Methods for preparing such dosage forms are known (see, for example, H. C. Ansel and N. G. Popovish, Pharmaceutical Dosage Forms and Drug Delivery Systems, 5th ed., Lea and Febiger (1990)). Dosage levels and requirements for the compounds of the present invention may be selected by those of ordinary skill in the art from available methods and techniques suitable for a particular patient. In some embodiments, dosage levels range from about 1-1000 mg/dose for a 70 kg patient. Although one dose per day may be sufficient, up to 5 doses per day may be given. Doses up to 2000 mg/day may be required. As the skilled artisan will appreciate, lower or higher doses may be required depending on particular factors. For instance, specific dosage and treatment regimens will depend on factors such as the patient's general health profile, the severity and course of the patient's disorder or disposition thereto, and the judgment of the treating physician.

In one embodiment, for example, multiple compounds of the present invention can be administered. Advantageously, such combination therapies utilize lower dosages of the conventional therapeutics, thus avoiding possible toxicity and adverse side effects incurred when those agents are used as monotherapies. Compounds of the invention may be physically combined with the conventional therapeutics or other adjuvants into a single pharmaceutical composition. Advantageously, the compounds may then be administered together in a single dosage form. In some embodiments, the pharmaceutical compositions comprising such combinations of compounds contain at least about 5%, but more preferably at least about 20%, of a compound of formula (I) (w/w) or a combination thereof. The optimum percentage (w/w) of a compound of the invention may vary and is within the purview of those skilled in the art. Alternatively, the compounds of the present invention and the conventional therapeutics or other adjuvants may be administered separately (either serially or in parallel). Separate dosing allows for greater flexibility in the dosing regimen.

In one embodiment, the compounds of the invention are administered in combination with a sodium-glucose cotransporter-2 (SGLT2) inhibitors. Nonlimiting examples of SGLT2 inhibitors include bexaglifloxin, canagliflozin, dapagliflozin, empagliflozin, and ertugliflozin. In one embodiment, the compounds of the invention are administered in combination with empagliflozin. In another embodiment, the compound of the invention are administered in combination with empagliflozin, wherein the compound of the invention is selected from the group consisting of Form I, Form X, and mixtures thereof. In another embodiment, empagliflozin is administered to the patient in a daily amount of 10 mg or 25 mg. In another embodiment, empagliflozin is administered to the patient once daily in an amount of 10 mg.

In one embodiment, the inventions relates to the methods of treatment or uses described above wherein the compound of the invention is administered to the patient once daily in amount of 3 mg, 10 mg or 20 mg; and empagliflozin is administered to the patient once daily in an amount of 10 mg.

In another embodiment, the inventions relates to the methods of treatment or uses described above wherein the compound of the invention is administered to the patient once daily in amount of 10 mg; and in another embodiment, the compound of the invention is administered to the patient in combination with empagliflozin, wherein the compound of the invention is administered once daily in amount of 10 mg and empagliflozin is administered once daily in an amount of 10 mg.

### EXAMPLES

A racemic mixture of Compound 1 and its S-enantiomer may be prepared as described in WO 2016/014736 and the enantiomers separated using chiral chromatography to provide Compound 1. The volatile components are removed to provide Compound 1 as a solid residue (“the solid residue of Compound 1”). Unless otherwise stated, this solid residue of Compound 1 was used as the starting material in the Examples described below.

### Example 1

### Preparation of Form I of Compound 1

### Preparation of Form I

The solid residue of Compound 1 is dissolved in a mixture of THF and water (75% THF/25% H2O (w/w)) at 55-60° C. Water is then added to reach a THF content of 57 wt %, and the solution is cooled to 40-45° C. The solution is then seeded with the solid residue of Compound 1 (prepared as described above) (1 wt. %), and the slurry is aged for 1 hr at 40° C.

Water is then added over 3 hrs to reach a THF content in the slurry of 33 wt % while maintaining a temperature of 40-45° C. Two heat cycles are then completed after cooling the slurry to RT. The two heating cycles go from room temperature to 50° C., and end at room temperature before isolation by filtration and vacuum drying at 50° C. for 12 hr under nitrogen bleed.

### Second (Alternate) Preparation of Form I

A solid residue of Compound 1 (3.2 g) is fully dissolved in 85% acetone in H₂O (w/w) (59 ml) at 55° C. with stirring. Water (10.3 ml) is then added to the solution and the solution is distilled under atmospheric pressure at 55-60° C. down to a volume of 45 ml. The resulting solution is then seeded with 30 mg of Form I (prepared as described above) and aged for 1 hr with stirring. Then 24 ml H₂O is added to the resulting slurry over 30 min and the slurry is aged for 30 mins. The slurry is then cooled to 20° C. over 2 hrs. The solids are collected by filtration, washed with H₂O, then dried under vacuum at 65° C. to provide Form I of Compound 1.

### Example 2

### Preparation of Form X

Form I (prepared as described above) is dissolved to saturation in nitromethane and heated to 80° C. for one hour. The solution is then placed in a refrigerator at 4° C. overnight and the resulting nitromethane solvate is isolated by filtration, air dried, and confirmed to be a nitromethane solvate of Compound 1 by XRPD. The nitromethane solvate is then slurried in MIBK for 5 days at room temperature with a magnetic stirrer, resulting in full conversion to Form X. The solid is then filtered and air dried to provide Form X.

### Alternate Preparation of Form X:

The same procedure described in Example 1 (Second Alternate Preparation of Form 1) is followed except the seeding is done with Form X. Acetone is added to the slurry after crystallization is complete to make a final solvent composition 50/50 acetone/H₂O by weight. The slurry is then wet milled with a circulating wet mill at room temperature until the solids are fully converted to Form X.

### Example 3

### Preparation of Form II

Form I (prepared as described below) is dissolved in IPA at 90° C. and rapidly cooled at 4° C. to provide Form II.

### First Alternate Preparation of Form II

Form I (prepared as described below) is dissolved in DMSO and added to water to provide Form II.

### Second Alternate Preparation of Form II

A solution of Form I in methanol (30 mg/g) is heat to 65° C., cooled to 5° C., and maintained at 5° C. without stirring for 3 hours to provide Form II.

### Example 4

### Preparation of Form III of Compound 1

Form I (prepared as described in Example 1) is dissolved in methyl isobutyl ketone at 82° C. and the solution is stirred for 1.25 hrs. The solution is then fast cooled to 4° C. in a refrigerator. The resulting slurry is left to stand for 12 hrs to provide Form III.

### Alternate Preparation of Form III

Form I (prepared as described in Example 1) is dissolved in a solution of MEK/Heptane and recrystallized at elevated temperature to provide Form III.

### Example 5

### Preparation of Form IV (Acetonitrile Solvate of Compound 1

A saturated acetonitrile of the solid residue of Compound 1 is heated to 65° C., the hot slurry is filtered, and the filtrate is placed in freezer at −20° C. overnight to provide the acetonitrile solvate (Form IV).

### Example 6

### Preparation of Form VI (Acetone Solvate of Compound 1)

A saturated acetone solution of Form I of Compound 1 (prepared as described in Example 1) is chilled to provide Form VI.

### Example 7

### Preparation of Form VII (THF Solvate of Compound 1)

A saturated THF solution of Form I of Compound 1 (prepared as described in Example 1) is chilled, and the resulting solids are collected and dried under air saturated with THF to provide Form VII.

### Example 8

### Preparation of Form IX (Toluene Solvate of Compound 1)

Form I of Compound I (prepared as described in Comparative Example 1) is Fully dissolved in toluene and crystallized out by cooling to provide Form IX, the hemi-toluene solvate.

### Alternate Preparation of Form IX.

The Amorphous form of Compound 1, as described in comparative example 9, is equilibrated in pure toluene to provide form IX, the hemi-toluene solvate.

### Comparative Example 9

### Preparation of Amorphous Form of Compound 1

Form I (prepared as described below) is treated with a mixture 1,4-dioxane/water 90/10% (v/v) to provide a solution concentration of 50 mg/mL. The solution is filtered to remove any seeds of Form 1. The solution is frozen in liquid nitrogen and lyophilized overnight to provide the amorphous form of Compound 1.

What is claimed is:

1. A solid crystalline form of Compound 1: 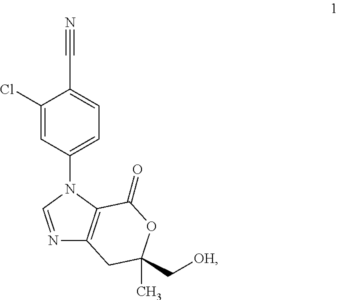 [SMILES: C[C@]1(CO)Cc2ncn(-c3ccc(C#N)c(Cl)c3)c2C(=O)O1]
wherein the solid crystalline form of Compound 1 is selected from the group consisting of:
i) Form I characterized by:
at least three XRPD peaks at 2θ angles selected from 12.6°, 14.1°, 14.9°, 21.3°, and 27.1°; or
¹³C solid-state nuclear magnetic resonance peaks at chemical shifts selected from 152.8 ppm, 152.2 ppm, 88.7 ppm, 68.8 ppm, and 19.9 ppm;
ii) Form II characterized by:
at least five XRPD peaks at 2θ angles selected from 11.5°, 13.5°, 14.7°, 14.9°, 16.1°, 17.1°, 19.7°, 20.5°, 21.5°, 23.0°, 24.5°, 25.1°, and 30.1°;
iii) Form III characterized by:
at least five XRPD peaks at 2θ angles selected from 6.8°, 13.7°, 17.9°, 20.8°, 23.8°, and 26.90;
iv) Form IV characterized by:
at least five XRPD peaks at 2θ angles selected 5.7°, 12.8°, 19.9°, 21.0°, 22.4°, 22.9°, 23.3°, and 27.6°;
v) Form VI characterized by:
at least five XRPD peaks at 2θ angles selected from 5.2°, 12.1° C., 19.7°, 20.7°, 24.0°, and 26.4°;
vi) Form VII characterized by:
at least five XRPD peaks at 2θ angles selected from 5.2°, 17.5°, 19.7°, 20.8°, 21.7°, 24.0°, and 26.4°;
(vii) Form IX characterized by:
at least five XRPD peaks at 2θ angles selected from 8.6°, 9.7°, 13.0°, 13.1°, 14.1°, 15.0°, 16.2°, 19.5°, 20.4°, 20.7°, 23.6°, 24.6°, 28.4°, and 30.0°; and
viii) Form X characterized by:
at least five XRPD peaks at 2θ angles selected from 9.9°, 12.9°, 15.5°, 20.5°, 21.8°, 26.3°, and 27.8°; or
¹³C solid-state nuclear magnetic resonance peaks at chemical shifts selected from 157.7 ppm, 151.4 ppm, 88.1 ppm, 69.6 ppm, and 20.6 ppm.

2. A pharmaceutical composition comprising any of Form I or Form X according to claim 1, optionally together with one or more inert carriers and/or diluents.

3. A method for treating and/or preventing a disease or disorder that is responsive to treatment with an inhibitor of aldosterone synthase, comprising administering to a patient in need thereof a pharmaceutically effective amount of Form I or Form X according to claim 1.

4. A method for preventing, slowing the progression of, delaying or treating chronic kidney disease, diabetic kidney disease, heart failure, heart failure with reduced ejection fraction (HFrEF), heart failure with preserved ejection fraction (HFpEF), heart failure with left ventricular ejection fraction≥40% (LVEF≥40%), heart failure with LVEF<40%, and/or resistant hypertension (rHPT), comprising administering a pharmaceutically effective amount of Form I or Form X according to claim 1 to a patient in need thereof.

5. The method according to claim 4, wherein Form I or Form X is orally administered in a daily amount of 0.1 to 100 mg; or 1 to 50 mg; or 1 to 25 mg; or 1 to 20 mg.

6. The method according to claim 5, wherein Form I or Form X is orally administered in a daily amount of 3 mg, or 10 mg, or 20 mg.

7. The method according to claim 6, further comprising administering Form I or Form X in combination with an SGLT2 inhibitor.

8. The method to claim 7, wherein the SGLT2 inhibitor is selected from the group consisting of bexaglifloxin, canagliflozin, dapagliflozin, empagliflozin and ertugliflozin.

9. The method according to claim 8, wherein the SGLT2 inhibitor is empagliflozin.

10. A method of producing a solid crystalline form of Compound 1 of claim 1, the method comprising:
(a) dissolving Compound 1 in a suitable solvent at elevated temperature,
(b) optionally, filtering the solution of step (i) to provide a filtered solution of Compound 1 (“the filtered first solution”),
(c) concentrating and/or cooling the solution of step (a) or the filtered solution of step (b) to provide a mixture, and
(d) isolating the solids from the mixture of step (c) to provide the solid crystalline form of Compound 1.
